(19) United States
(12) Patent Application Publication
(10) Pub. No.: US 2002/0183519 A1
(43) Pub. Date: Dec. 5, 2002

(54) Antithrombotic carboxylic acid amides

(76) Inventors: Herbert Nar, Ochsenhausen (DE); Henning Priepke, Warthausen (DE); Uwe Ries, Biberach (DE); Jean Marie Stassen, Leuven (BE); Wolfgang Wienen, Biberach/Rissegg (DE)

Correspondence Address: BOEHRINGER INGELHEIM CORPORATION, 900 RIDGEBURY ROAD, P. O. BOX 368, RIDGEFIELD, CT, 06877, US

(21) Appl. No.: 10/096,526

(22) Filed: Mar. 11, 2002

Related U.S. Application Data
(60) Provisional application No. 60/280,449, filed on Mar. 30, 2001.

(30) Foreign Application Priority Data
Mar. 13, 2001 (DE) DE 101 11 842.2

(51) Int. Cl. C07D 417/02; C 07D 4 9/02; C 07D 4 3/02; C 07D 4 1/02

(52) U.S. Cl. 544/238; 544/405; 546/268.1; 544/333; 548/202; 549/59

(57) ABSTRACT

Carboxylic acid amides of formula

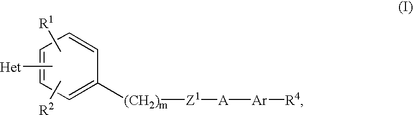

having antithrombotic activity and a factor Xa-inhibiting activity. Exemplary are:

2-(5-amidino-2-hydroxy-phenyl)-N-[3-methyl-4-(2-tert-butoxycarbonylaminomethyl -benzimidazol-1-yl)-phenyl]-acetamide;

2-(5-amidino-2-hydroxy-phenyl)-N-[3-methyl-4-(2-aminomethyl-benzimidazol-1-yl)-phenyl]-acetamide; and

2-(5-amidino-2-hydroxy-phenyl)-N-[4-(4,5-dimethyl-2-oxo-2,3-dihydroimidazol-1-yl) -3-methyl-phenyl]-acetamide.

### RELATED APPLICATIONS

Benefit of U.S. Provisional Application Serial No. 60/280,449, filed on Mar. 30, 2001, is hereby claimed.

### DESCRIPTION OF THE INVENTION

The invention relates to carboxylic acid amides of formula

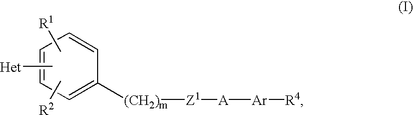

their tautomers, stereoisomers, mixtures thereof, the prodrugs thereof, the derivatives thereof which contain a group which is negatively charged under physiological conditions instead of a carboxy group, and the salts thereof, particularly the physiologically acceptable salts thereof with inorganic or organic acids or bases which have valuable properties.

German Patent Application DE 199 37 494 describes similar carboxylic acid amides, although they do not have any directly connected heterocyclic groups.

The aim of the present application was to provide compounds having comparable effects to those described in DE 199 37 494 but which can be made into better formulations thanks to their physicochemical properties.

This aim was achieved according to the invention by preparing the new compounds of formula I which are characterised in particular by improved solubility.

The compounds of the above general formula I wherein R⁴ denotes a cyano group, are valuable intermediate products for preparing the corresponding compounds of general formula I wherein R⁴ denotes an aminomethyl group or an amidino group optionally substituted by one or two C₁₋₃-alkyl groups. The compounds of the above formula I with the exception of those compounds wherein R⁴ denotes a cyano group as well as their tautomers, stereoisomers, mixtures thereof, the prodrugs thereof, the derivatives thereof which contain a group which is negatively charged under physiological conditions instead of a carboxy group, and the salts thereof, particularly the physiologically acceptable salts thereof with inorganic or organic acids, and the stereoisomers thereof have valuable pharmacological properties, particularly an antithrombotic activity and a factor Xa-inhibiting activity.

The present application thus relates to the new compounds of the above general formula I and their preparation, pharmaceutical compositions containing the pharmacologically active compounds, the preparation and use thereof.

In the above formula I:

m denotes 0 or 1,

A denotes a straight-chain C₁₋₃-alkylene group wherein

one or two hydrogen atoms independently of one another may each be replaced by a C₁₋₃ alkyl group or

a hydrogen atom may be replaced by the group —(CH₂)ₚ—R_{f}, wherein

p denotes one of the numbers 0, 1, 2 or 3 and

R_{f} denotes a hydroxycarbonyl, C₁₋₃-alkoxycarbonyl, aminocarbonyl, C₁₋₃-alkylaminocarbonyl, C₁₋₃-(dialkyl)-aminocarbonyl or C₃₋₇-cycloalkylamino-carbonyl group,

Ar denotes a phenylene or naphthylene group optionally substituted by a fluorine, chlorine or bromine atom, by a carboxy, carboxy-C₁₋₃-alkyl, carboxy-C₁₋₃-alkoxy, alkoxycarbonyl-C₁₋₃-alkoxy, trifluoromethyl, C₁₋₃-alkyl, hydroxy, C₁₋₃-alkoxy, phenyl-C₁₋₃-alkoxy, amino, C₁₋₃-alkylamino or di-(C₁₋₃-alkyl)-amino group, wherein the phenylene group may be substituted by another fluorine, chlorine or bromine atom or by another C₁₋₃-alkyl group; or

a thienylene, thiazolylene, pyridinylene, pyrimidinylene, pyrazinylene or pyridazinylene group optionally substituted in the carbon skeleton by a C₁₋₃-alkyl group,

Het denotes a 5 or 6-membered heterocyclic group optionally substituted by one, two or three substituents and bound via a carbon or nitrogen atom, wherein these substituents are selected from among ═O, a fluorine, chlorine or bromine atom, a trifluoromethyl, C₁₋₃-alkyl, hydroxy, hydroxy-C₁₋₃-alkyl, C₁₋₃-alkoxy, phenyl-C₁₋₃-alkoxy, C₁₋₃-alkylaminocarbonyl, C₁₋₃-dialkylaminocarbonyl, C₃₋₇-cycloalkylaminocarbonyl, C₁₋₃-alkylaminocarbonyl-(CH₂)ₒ—C₁₋₃-alkoxycarbonyl or C₁₋₃-alkoxycarbonyl-(CH₂)ₒ— group and the group of formula

RₐR_{b}N—(CH₂)ₒ,

wherein

Rₐ and R_{b} independently of one another each denote a hydrogen atom or a C₁₋₃-alkyl, aminocarbonyl, C₁₋₄-alkyloxycarbonyl, amidino, C₁₋₃-alkylCOHNC(NH), C₁₋₃-alkylC(NH), imidazol-2-yl or 4,5-dihydroimidazol-2-yl-group, or Rₐ and R_{b} taken together form a C₂₋₅-alkylenediyl group, and o denotes 0 or 1,

wherein a phenyl or C₄₋₈-cycloalkyl ring may be fused to the abovementioned 5- or 6-membered heterocyclic group via two adjacent cyclic atoms and the bicyclic heterocyclyl groups thus formed may be bound via the heterocyclic or carbocyclic moiety,

Z¹ denotes a —CO—NR³ or —NR³—CO— group;

R¹ denotes a hydrogen, fluorine, chlorine or bromine atom, a hydroxy group or a C₁₋₃-alkyl or C₁₋₃-alkoxy group optionally substituted by one or more fluorine atoms,

R² denotes a hydrogen atom or a C₁₋₃-alkyl group,

R³ denotes a hydrogen atom or a C₁₋₃-alkyl group optionally substituted by a carboxy group, and

R⁴ denotes a cyano group, an aminomethyl group or an amidino group optionally substituted by one or two C₁₋₃-alkyl groups.

The compounds of formula I wherein the groups have the following meanings are preferred:

m denotes the number 0 or 1,

A denotes a methylene group wherein

one or two hydrogen atoms independently of one another may each be replaced by a C₁₋₃ alkyl group or a hydrogen atom may be replaced by the group —(CH₂)ₚ—R_{f}, wherein

p denotes one of the numbers 0, 1, 2 or 3 and

R_{f} denotes a hydroxycarbonyl, C₁₋₃-alkoxycarbonyl, aminocarbonyl, C₁₋₃-alkylaminocarbonyl, C₁₋₃-(dialkyl)-aminocarbonyl or C₃₋₇-cycloalkylamino-carbonyl group,

Ar denotes a phenylene group optionally substituted by a fluorine, chlorine or bromine atom, by a methyl, hydroxy, methoxy or benzyloxy group which may be substituted by another methyl group;

Het denotes a 5- or 6-membered heterocyclic group with two nitrogen atoms optionally substituted by one, two or three substituents and bound via a carbon or nitrogen atom, wherein these substituents are selected from among ═O, a C₁₋₃-alkyl, hydroxy, C₁₋₃-alkoxy, phenyl-C₁₋₃-alkoxy, C₁₋₃-alkylaminocarbonyl, C₁₋₃-dialkylaminocarbonyl, C₃₋₇-cycloalkylaminocarbonyl or C₁₋₃-alkoxycarbonyl group and the group of formula

RₐR_{b}N—(CH₂)ₒ—,

wherein

Rₐ and R_{b} independently of one another each denote a hydrogen atom, a tert.butyloxycarbonyl group or a C₁₋₃-alkyl group, or taken together form a C₃₋₅-alkylenediyl group, and

o denotes 0 or 1,

wherein a phenyl or C₄₋₈-cycloalkyl ring may be fused to the abovementioned 5- or 6-membered heterocyclic group via two adjacent carbon atoms or a carbon atom and an adjacent nitrogen atom and the bicyclic heterocyclyl groups thus formed may be bound via the heterocyclic or carbocyclic moiety,

Z¹ denotes a —CO—NR³ or —NR³—CO— group;

R¹ denotes a hydrogen, fluorine, chlorine or bromine atom, a methyl, trifluoromethyl, difluoromethyl, hydroxy or methoxy group,

R² denotes a hydrogen atom or a methyl group,

R³ denotes a hydrogen atom or a methyl or ethyl group optionally substituted by a carboxy or C₁₋₃-alkoxycarbonyl group, and

R⁴ denotes a cyano group, an aminomethyl group or an amidino group.

Particularly preferred are the compounds of formula I wherein the groups have the following meanings:

m denotes the number 0,

A denotes a methylene group wherein

a hydrogen atom may be replaced by a C₁₋₃ alkyl group or by the group —(CH₂)ₚ—R_{f}, wherein

p denotes one of the numbers 0, 1, 2 or 3 and

R_{f} denotes a hydroxycarbonyl, C₁₋₃-alkoxycarbonyl, aminocarbonyl, C₁₋₃-alkylaminocarbonyl or C₁₋₃-(dialkyl)-aminocarbonyl group,

Ar denotes a phenylene group optionally substituted by a methyl, hydroxy, methoxy or benzyloxy group,

Het denotes an imidazolyl, pyrazolyl, pyridyl or pyrimidyl group optionally substituted by a substituent selected from among the C₁₋₃-alkyl, hydroxy, C₁₋₃-alkylaminocarbonyl or C₁₋₃-alkoxycarbonyl group and the group of formula

RₐR_{b}N—(CH₂)ₒ,

wherein

Rₐ and R_{b} independently of one another each denote a hydrogen atom or a C₁₋₃-alkyl group, or Rₐ and R_{b} taken together form a C₂₋₅-alkylenediyl group, and

o denotes 0 or 1,

wherein a phenyl or C₅₋₆-cycloalkyl ring is fused to this group via two adjacent carbon atoms and the bicyclic heterocyclyl groups thus formed are bound via the heterocyclic moiety,

Z¹ denotes a —CO—NR³ or —NR³—CO— group;

R¹ denotes a hydrogen, fluorine, chlorine or bromine atom or a methyl or trifluoromethyl group,

R² denotes a hydrogen atom,

R³ denotes a hydrogen atom or a methyl or ethyl group substituted by a carboxy, methoxycarbonyl or ethoxycarbonyl group, and

R⁴ denotes an aminomethyl or amidino group.

Preferred compounds of formula I are those wherein —Ar—R⁴ denotes a group of formula

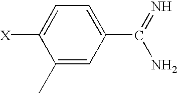

wherein X represents a hydrogen atom, a halogen atom or a hydroxy group.

Particularly preferred are those compounds of formula I wherein —Ar—R⁴ denotes a 5-amidino-2-hydroxyphenyl group.

Another preferred embodiment of the invention consists of the compounds of formula IA,

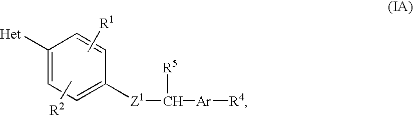

wherein R¹, R², R³, R⁴, Ar, Het and Z¹ are as hereinbefore defined, and

R⁵ denotes a hydrogen atom, a C₁₋₃ alkyl group or a group of formula —(CH₂)ₚ—R_{f}, wherein

p denotes one of the numbers 0, 1, 2 or 3 and

R_{f} denotes a hydroxycarbonyl, C₁₋₃-alkoxycarbonyl, aminocarbonyl, C₁₋₃-alkylaminocarbonyl, C₁₋₃-(dialkyl)-aminocarbonyl or C₃₋₇-cycloalkylamino-carbonyl group.

By the term “amidino” or “carboxamimidoyl” as used hereinbefore and hereinafter for the group “R⁴” is meant a group of formula

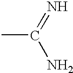

By the term “heterocyclic group” as used hereinbefore and hereinafter for the group “Het” is meant saturated, unsaturated or aromatic 5- or 6-membered rings which contain, apart from carbon atoms, at least one heteroatom selected from among nitrogen, oxygen and sulphur. The following heterocyclyl groups are preferred:

5-membered heteroaryl groups which contain

an optionally substituted imino group, an oxygen or sulphur atom,

an optionally substituted imino group and an oxygen, sulphur or nitrogen atom,

an optionally substituted imino group and two nitrogen atoms or

an oxygen or sulphur atom and two nitrogen atoms,

6-membered heteroaryl groups which contain

one or two nitrogen atoms, or

saturated 5- or 6-membered heterocyclyl groups which contain

one or two nitrogen atoms,

a nitrogen atom and an oxygen or sulphur atom, or

one or two oxygen or sulphur atoms,

wherein a phenyl ring or cycloalkyl ring may be fused to the abovementioned 5- or 6-membered heterocyclyl groups via two adjacent cyclic atoms and the bicyclic heterocyclyl groups thus formed may be bound via the heterocyclic or carbocyclic moiety.

The unsubstituted or monosubstituted phenyl groups mentioned in the definition of the abovementioned groups, or the unsubstituted or monosubstituted phenyl moieties contained in these groups, as well as the abovementioned heterocyclyl groups may additionally be substituted at a carbon atom in each case by a fluorine, chlorine or bromine atom, by a trifluoromethyl, C₁₋₃-alkyl or C₁₋₃-alkoxy group, unless otherwise stated.

The carboxy groups mentioned in the definition of the abovementioned groups may be replaced by a group which may be converted in vivo into a carboxy group or by a group which is negatively charged under physiological conditions, and moreover the amino and imino groups mentioned in the definition of the abovementioned groups may be substituted by a group which can be cleaved in vivo. Such groups are described for example in WO 98/46576 and by N. M. Nielsen et al. in International Journal of Pharmaceutics 39, 75-85 (1987).

By a group which can be converted in vivo into a carboxy group is meant, for example, a hydroxymethyl group, a carboxy group esterified with an alcohol wherein the alcohol moiety is preferably a C₁₋₆-alkanol, a phenyl-C₁₋₃-alkanol, a C₃₋₉-cycloalkanol, while a C₅₋₈-cycloalkanol may additionally be substituted by one or two C₁₋₃-alkyl groups, a C₅₋₈-cycloalkanol wherein a methylene group in the 3 or 4 position is replaced by an oxygen atom or by an imino group optionally substituted by a C₁₋₃-alkyl, phenyl-C₁₋₃-alkyl, phenyl-C₁₋₃-alkoxycarbonyl or C₂₋₆-alkanoyl group and the cycloalkanol moiety may additionally be substituted by one or two C₁₋₃-alkyl groups, a C₄₋₇-cycloalkenol, a C₃₋₅-alkenol, a phenyl-C₃₋₅-alkenol, a C₃₋₅-alkynol or phenyl-C₃₋₅-alkynol with the proviso that no bonds to the oxygen atom start from a carbon atom which carries a double or triple bond, a C₃₋₈-cycloalkyl-C₁₋₃-alkanol, a bicycloalkanol with a total of 8 to 10 carbon atoms which may additionally be substituted in the bicycloalkyl moiety by one or two C₁₋₃-alkyl groups, a 1,3-dihydro-3-oxo-1-isobenzofuranol or an alcohol of formula

Rₓ—CO—O—(R_{y}CR_{z})—OH,

wherein

Rₓ denotes a C₁₋₈-alkyl, C₅₋₇-cycloalkyl, phenyl or phenyl-C₁₋₃-alkyl group,

R_{y} denotes a hydrogen atom, a C₁₋₃-alkyl, C₅₋₇-cycloalkyl or phenyl group and

R_{z} denotes a hydrogen atom or a C₁₋₃-alkyl group, by a group which is negatively charged under physiological conditions is meant, for example, a tetrazol-5-yl, phenylcarbonylaminocarbonyl, trifluoromethylcarbonylaminocarbonyl, C₁₋₆-alkylsulphonylamino, phenylsulphonylamino, benzylsulphonylamino, trifluoromethylsulphonylamino, C₁₋₆-alkylsulphonylaminocarbonyl, phenylsulphonylaminocarbonyl, benzylsulphonylaminocarbonyl or perfluoro-C₁₋₆-alkylsulphonylaminocarbonyl group and by a group which can be cleaved in vivo from an imino or amino group is meant, for example, a hydroxy group, an acyl group such as a phenylcarbonyl group optionally mono- or disubstituted by fluorine, chlorine, bromine or iodine atoms, by C₁₋₃-alkyl or C₁₋₃-alkoxy groups, while the substituents may be identical or different, a pyridinoyl group or a C₁₋₁₆-alkanoyl group such as the formyl, acetyl, propionyl, butanoyl, pentanoyl or hexanoyl group, a 3,3,3-trichloropropionyl or allyloxycarbonyl group, a C₁₋₁₆-alkoxycarbonyl or C₁₋₁₆-alkylcarbonyloxy group, wherein hydrogen atoms may be wholly or partially replaced by fluorine or chlorine atoms such as the methoxycarbonyl, ethoxycarbonyl, propoxycarbonyl, isopropoxycarbonyl, butoxycarbonyl, tert.butoxycarbonyl, pentoxycarbonyl, hexoxycarbonyl, octyloxycarbonyl, nonyloxycarbonyl, decyloxycarbonyl, undecyloxycarbonyl, dodecyloxycarbonyl, hexadecyloxycarbonyl, methylcarbonyloxy, ethylcarbonyloxy, 2,2,2-trichloroethylcarbonyloxy, propylcarbonyloxy, isopropylcarbonyloxy, butylcarbonyloxy, tert.butylcarbonyloxy, pentylcarbonyloxy, hexylcarbonyloxy, octylcarbonyloxy, nonylcarbonyloxy, decylcarbonyloxy, undecylcarbonyloxy, dodecylcarbonyloxy or hexadecylcarbonyloxy group, a phenyl-C₁₋₆-alkoxycarbonyl group such as the benzyloxycarbonyl, phenylethoxycarbonyl or phenylpropoxycarbonyl group, a 3-amino-propionyl group wherein the amino group may be mono- or disubstituted by C₁₋₆-alkyl or C₃₋₇-cycloalkyl groups and the substituents may be identical or different, a C₁₋₃-alkylsulphonyl-C₂₋₄-alkoxycarbonyl, C₁₋₃-alkoxy-C₂₋₄-alkoxy-C₂₋₄-alkoxycarbonyl, Rₓ-CO—O—(R_{y}CR_{z})—O—CO—, C₁₋₆-alkyl-CO—NH—(R_{d}CRₑ)—O—CO— or C₁₋₆-alkyl-CO—O—(R_{d}CRₑ)-(R_{d}CRₑ)—O—CO— group, wherein Rₓ to R_{z} are as hereinbefore defined,

R_{d} and Rₑ, which may be identical or different, denote hydrogen atoms or C₁₋₃-alkyl groups.

Moreover, the saturated alkyl and alkoxy moieties containing more than 2 carbon atoms mentioned in the definitions above also include the branched isomers thereof such as the isopropyl, tert.butyl, isobutyl group, etc.

Particularly preferred compounds of general formula I are those wherein

Het is a heterocyclic group bound in the 4-position of the phenyl group of formula I selected from among groups (a) to (g),

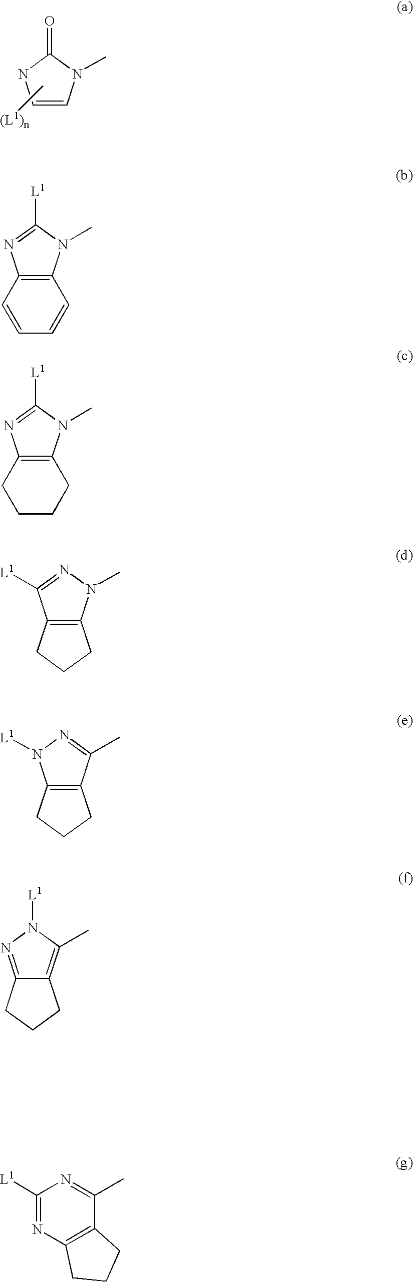

wherein

L¹ denotes a hydrogen, fluorine, chlorine or bromine atom, or a trifluoromethyl, C₁₋₃-alkyl, hydroxy, hydroxy-C₁₋₃-alkyl, C₁₋₃-alkoxy, phenyl-C₁₋₃-alkoxy, C₁₋₃-alkylaminocarbonyl, C₁₋₃-dialkylaminocarbonyl, C₃₋₇-cycloalkylaminocarbonyl, C₁₋₃-alkylaminocarbonyl-(CH₂)ₒ—C₁₋₃-alkoxycarbonyl, C₁₋₃-alkoxycarbonyl-(CH₂)ₒ— group or the group of formula

RₐR_{b}N—(CH₂)ₒ—,

wherein

Rₐ and R_{b} independently of one another each denote a hydrogen atom or a C₁₋₃-alkyl, aminocarbonyl, C₁₋₄-alkyloxycarbonyl, amidino, C₁₋₃-alkylCOHNC(NH), C₁₋₃-alkylC(NH), imidazol-2-yl or 4,5-dihydroimidazol-2-yl group, or Rₐ and R_{b} taken together form a C₂₋₅-alkylenediyl group, and o denotes 0 or 1.

Also preferred are compounds of formula I wherein

R¹ denotes a hydrogen atom or a substituent bound in the 3 position of the phenyl group in formula I selected from among fluorine, chlorine, bromine, C₁₋₃-alkyl and trifluoromethyl, especially chlorine, methyl or trifluoromethyl;

R² denotes a hydrogen atom or a C₁₋₃-alkyl group bound in the 2 position of the phenyl group in formula I, particularly hydrogen,

R³ denotes a hydrogen atom and

Ar denotes a phenyl group optionally substituted by a hydroxy group bound in the 2 position, and

R₅ denotes an amidino group bound in the 5 position by Ar and optionally substituted by one or two C₁₋₃-alkyl groups, a C₁₋₆-alkoxy-carbonyl or phenylcarbonyl group, the isomers and the salts thereof.

The following preferred compounds are mentioned by way of example:

2-(5-amidino-2-hydroxy-phenyl)-N-[3-methyl-4-(2-tert-butoxycarbonylaminomethyl-benzimidazol-1-yl)-phenyl]-acetamide

2-(5-amidino-2-hydroxy-phenyl)-N-[3-methyl-4-(2-aminomethyl-benzimidazol-1-yl)-phenyl]-acetamide

2-(5-amidino-2-hydroxy-phenyl)-N-[4-(4,5-dimethyl-2-oxo-2,3-dihydroimidazol-1-yl)-3-methyl-phenyl]-acetamide

2-(5-amidino-2-hydroxy-phenyl)-N-[4-(benzimidazol-1-yl)-3-methyl-phenyl]-acetamide

2-(5-amidino-2-hydroxy-phenyl)-N-[3-methyl-4-(2-methyl-benzimidazol-1-yl)-phenyl]-acetamide

2-(5-amidino-2-hydroxy-phenyl)-N-[3-methyl-4-((2-pyrrolidin-1-yl)-benzimidazol-1-yl)-phenyl]-acetamide

2-(5-amidino-2-hydroxy-phenyl)-N-[3-methyl-4-(2-dimethylamino-benzimidazol-1-yl)-phenyl]-acetamide

2-(5-amidino-2-hydroxy-phenyl)-N-[3-methyl-4-(4,5,6,7-tetrahydro-benzimidazol-1-yl)-phenyl]-acetamide

N-[2-(5-amidino-2-hydroxy-benzyl)]-N-3-trifluoromethyl-4-(4,5,6,7-tetrahydro-benzimidazol-1-yl)-benzamide

N-[1-(5-amidino-2-hydroxy-phenyl)-ethyl]-N-3-trifluoromethyl-4-(4,5,6,7-tetrahydro-benzimidazol-1-yl)-benzamide

2-(5-amidino-2-hydroxy-phenyl)-N-[3-methyl-4-(4,5,6,7-tetrahydro-benzimidazol-1-yl)-phenyl]-propionamide

2-(5-amidino-2-hydroxy-phenyl)-N-[3-methyl-4-(4,5,6,7-tetrahydro-benzimidazol-1-yl)-phenyl]-acetamide

2-(5-amidino-2-hydroxy-phenyl)-N-[3-methyl-4-(3-ethoxycarbonyl-1,4,5,6-tetrahydro-cyclopentapyrazol-1-yl)-phenyl]-acetamide

2-(5-amidino-2-hydroxy-phenyl)-N-[4-(3-methyl-1,4,5,6-tetrahydro-cyclopentapyrazol-1-yl)-phenyl]-acetamide

N-(5-amidino-2-hydroxy-benzyl)-3-methyl-4-(3-methyl-1,4,5,6-tetrahydro-cyclopentapyrazol-1-yl)]-benzamide

2-(5-amidino-2-hydroxy-phenyl)-N-[3-methyl-4-(1-methyl-1,4,5,6-tetrahydro-cyclopentapyrazol-3-yl)-phenyl]-acetamide

2-(5-amidino-2-hydroxy-phenyl)-N-[3-methyl-4-(2-methyl-2,4,5,6-tetrahydro-cyclopentapyrazol-3-yl)-phenyl]-acetamide

2-(5-amidino-2-hydroxy-phenyl)-N-[3-methyl-4-(1,4,5,6-tetrahydro-cyclopentapyrazol-3-yl)-phenyl]-acetamide

2-(5-amidino-2-hydroxy-phenyl)-N-[4-(3-dimethylaminomethyl-1,4,5,6-tetrahydro-cyclopentapyrazol-1-yl)-3-methyl-phenyl]-acetamide

2-(5-amidino-2-hydroxy-phenyl)-N-[4-(6,7-dihydro-5H-cyclopentapyrimidin-4-yl)-3-methyl-phenyl]-acetamide

N-(5-amidino-2-hydroxy-benzyl)-3-trifluoromethyl-4-(1-methyl-1,4,5,6-tetrahydro-cyclopentapyrazol-3-yl)-benzamide

N-(5-amidino-2-hydroxy-benzyl)-3-trifluoromethyl-4-(3-methylaminocarbonyl-1,4,5,6-tetrahydro-cyclopentapyrazol-1-yl)-benzamide

2-(5-amidino-2-hydroxy-phenyl)-N-[3-trifluoromethyl-4-(4,5,6,7-tetrahydro-benzimidazol-1-yl)-phenyl]-acetamide

2-(5-amidino-2-hydroxy-phenyl)-N-[3-trifluoromethyl-4-(4,5,6,7-tetrahydro-benzimidazol-1-yl)-phenyl]-propionamide

N-(5-amidino-2-hydroxy-benzyl)-N-3-trifluoromethyl-4-(4,5,6,7-tetrahydro-benzimidazol-1-yl)-benzamide

N-[1-(5-amidino-2-hydroxy-phenyl)-ethyl]-3-trifluoromethyl-4-(4,5,6,7-tetrahydro-benzimidazol-1-yl)-benzamide

in which any amidino group present may additionally be substituted by a C₁₋₆-alkoxy-carbonyl or phenylcarbonyl group, and the salts thereof.

According to the invention, the compounds of general formula I are prepared by methods known per se, for example by:

a) reacting a compound of formula II

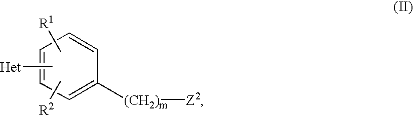

wherein

R¹, R², Het and m are as hereinbefore defined, with a compound of formula III

Z³—A—Ar—R⁴ (III)

wherein

A, Ar, R⁴ and n are as hereinbefore defined, wherein one of the groups Z² and Z³ denotes an amino group of formula —NR³H, and the other group Z² or Z³ denotes a carboxylate group of formula —COOH or the reactive derivatives thereof; or

b) to prepare a compound of formula I wherein R⁴ denotes an amidino group which may be substituted by one or two C₁₋₃-alkyl groups, reacting a compound of formula

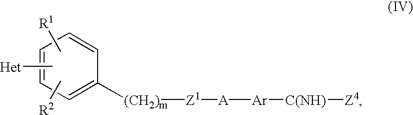

optionally formed in the reaction mixture wherein

R¹, R², Het, A, Ar, m and n are as hereinbefore defined and

Z⁴ denotes an alkoxy, aralkoxy, alkylthio or aralkylthio group, with an amine of general formula

H—R⁶NR⁷ (V),

wherein

R⁶ and R⁷, which may be identical or different, each denote a hydrogen atom or a C₁₋₃-alkyl group, or with the salts thereof; and subsequently, if desired, converting a compound of formula I thus obtained which contains an amino or imino group into a corresponding acyl compound of formula I by means of a corresponding acyl derivative, and/or converting a compound of formula I thus obtained which contains an esterified carboxy group into a corresponding carboxylic acid of formula I by hydrolysis, and/or converting a compound of formula I thus obtained which contains a carboxy group into a corresponding ester by esterification and/or cleaving any protecting groups used to protect reactive groups during the reactions, and/or resolving a compound of formula I thus obtained into the stereoisomers thereof, and/or converting a compound of formula I thus obtained into the salts thereof, particularly for pharmaceutical use into the physiologically acceptable salts thereof with an inorganic or organic acid or base.

The acylation (a) is conveniently carried out with a corresponding halide or anhydride in a solvent such as methylene chloride, chloroform, carbon tetrachloride, ether, tetrahydrofuran, dioxane, benzene, toluene, acetonitrile or sulpholane optionally in the presence of an inorganic or organic base at temperatures between −20 and 200° C., but preferably at temperatures between −10 and 160° C.

The acylation may however also be carried out with the free acid optionally in the presence of an acid-activating agent or a dehydrating agent, e.g. in the presence of isobutyl chloroformate, thionyl chloride, trimethylchlorosilane, hydrogen chloride, sulphuric acid, methanesulphonic acid, p-toluenesulphonic acid, phosphorus trichloride, phosphorus pentoxide, N,N′-dicyclohexylcarbodiimide, N,N′-dicyclohexyl-carbodiimide/N-hydroxysuccinimide or 1-hydroxy-benzotriazole, N,N′-carbonyldiimidazole or N,N′-thionyldiimidazole or triphenylphosphine/carbon tetrachloride, at temperatures between −20 and 200° C., but preferably at temperatures between −10 and 160° C.

The reaction (b) is conveniently carried out in a solvent such as methanol, ethanol, n-propanol, tetrahydrofuran or dioxane at temperatures between 0 and 150° C., preferably at temperatures between 0 and 80° C., with an amine of general formula V or with a corresponding acid addition salt such as for example ammonium carbonate or ammonium acetate.

A compound of general formula IV is obtained for example by reacting a corresponding cyano compound with a corresponding alcohol such as methanol, ethanol, n-propanol, isopropanol or benzyl alcohol in the presence of an acid such as hydrochloric acid or by reacting a corresponding amide with a trialkyloxonium salt such as triethyloxonium-tetrafluoroborate in a solvent such as methylene chloride, tetrahydrofuran or dioxane at temperatures between 0 and 50° C., but preferably at 20° C., or a corresponding nitrile with hydrogen sulphide conveniently in a solvent such as pyridine or dimethylformamide and in the presence of a base such as triethylamine and subsequently alkylating the thioamide formed with a corresponding alkyl or aralkyl halide.

The subsequent hydrolysis is conveniently carried out either in the presence of an acid such as hydrochloric acid, sulphuric acid, phosphoric acid, acetic acid, trichloroacetic acid, trifluoroacetic acid or mixtures thereof or in the presence of a base such as lithium hydroxide, sodium hydroxide or potassium hydroxide in a suitable solvent such as water, water/methanol, water/ethanol, water/isopropanol, methanol, ethanol, water/tetrahydrofuran or water/dioxane and the subsequent decarboxylation is carried out in the presence of an acid as hereinbefore described at temperatures between −10 and 120° C., e.g. at temperatures between ambient temperature and the boiling temperature of the reaction mixture.

The subsequent esterification is carried out with a corresponding alcohol, conveniently in a solvent or mixture of solvents such as methylene chloride, benzene, toluene, chlorobenzene, tetrahydrofuran, benzene/tetrahydrofuran or dioxane, but preferably in an excess of the alcohol used, optionally in the presence of an acid such as hydrochloric acid or in the presence of a dehydrating agent, e.g. in the presence of isobutyl chloroformate, thionyl chloride, trimethylchlorosilane, hydrochloric acid, sulphuric acid, methanesulphonic acid, p-toluenesulphonic acid, phosphorus trichloride, phosphorus pentoxide, N,N′-dicyclohexylcarbodiimide,N,N′-dicyclohexylcarbodiimide/N-hydroxysuccinimide, N,N′-carbonyldiimidazole or N,N′-thionyldiimidazole, triphenylphosphine/carbon tetrachloride or triphenyl-phosphine/diethyl azodicarboxylate, optionally in the presence of a base such as potassium carbonate, N-ethyl-diisopropylamine or N,N-dimethylamino-pyridine, conveniently at temperatures between 0 and 150° C., preferably at temperatures between 0 and 80° C., or with a corresponding halide in a solvent such as methylene chloride, tetrahydrofuran, dioxane, dimethylsulphoxide, dimethylformamide or acetone optionally in the presence of a reaction accelerator such as sodium or potassium iodide and preferably in the presence of a base such as sodium carbonate or potassium carbonate or in the presence of a tertiary organic base such as N-ethyl-diisopropylamine or N-methyl-morpholine, which may also simultaneously serve as the solvent, or optionally in the presence of silver carbonate or silver oxide at temperatures between −30 and 100° C., but preferably at temperatures between −10 and 80° C.

In the reactions described hereinbefore, any reactive groups present such as hydroxy, carboxy, amino, alkylamino or imino groups may be protected during the reaction by conventional protecting groups which are cleaved again after the reaction.

For example, a protecting group for a hydroxy group may be a methoxy, benzyloxy, trimethylsilyl, acetyl, benzoyl, tert-butyl, trityl, benzyl or tetrahydropyranyl group, protecting groups for a carboxy group may be a trimethylsilyl, methyl, ethyl, tert.butyl, benzyl or tetrahydropyranyl group and protecting groups for an amino, alkylamino or imino group may be an acetyl, trifluoroacetyl, benzoyl, ethoxycarbonyl, tert-butoxycarbonyl, benzyloxycarbonyl, benzyl, methoxybenzyl or 2,4-dimethoxybenzyl group and additionally, for the amino group, a phthalyl group.

Any protecting group used is optionally subsequently cleaved for example by hydrolysis in an aqueous solvent, e.g. in water, isopropanol/water, acetic acid/water, tetrahydrofuran/water or dioxane/water, in the presence of an acid such as trifluoroacetic acid, hydrochloric acid or sulphuric acid or in the presence of an alkali metal base such as lithium hydroxide, sodium hydroxide or potassium hydroxide or by ether splitting, e.g. in the presence of iodotrimethylsilane, at temperatures between 0 and 100° C., preferably at temperatures between 10 and 50° C.

However, a benzyl, methoxybenzyl or benzyloxycarbonyl group is cleaved, for example, hydrogenolytically, e.g. with hydrogen in the presence of a catalyst such as palladium/charcoal in a solvent such as methanol, ethanol, ethyl acetate, dimethylformamide, dimethylformamide/acetone or glacial acetic acid optionally with the addition of an acid such as hydrochloric acid at temperatures between 0 and 50° C., but preferably at ambient temperature, and at a hydrogen pressure of 1 to 7 bar, but preferably 3 to 5 bar.

A methoxybenzyl group may also be cleaved in the presence of a oxidising agent such as cerium(IV)ammonium nitrate in a solvent such as methylene chloride, acetonitrile or acetonitrile/water at temperatures between 0 and 50° C., but preferably at ambient temperature.

A methoxy group is conveniently cleaved in the presence of boron tribromide in a solvent such as methylene chloride at temperatures between −35 and −25° C.

A 2,4-dimethoxybenzyl group, however, is preferably cleaved in trifluoroacetic acid in the presence of anisole.

A tert.butyl or tert.butyloxycarbonyl group is preferably cleaved by treating with an acid such as trifluoroacetic acid or hydrochloric acid, optionally using a solvent such as methylene chloride, dioxane or ether.

A phthalyl group is preferably cleaved in the presence of hydrazine or a primary amine such as methylamine, ethylamine or n-butylamine in a solvent such as methanol, ethanol, isopropanol, toluene/water or dioxane at temperatures between 20 and 50° C.

An allyloxycarbonyl group is cleaved by treating with a catalytic amount of tetrakis-(triphenylphosphine)-palladium(O), preferably in a solvent such as tetrahydrofuran and preferably in the presence of an excess of a base such as morpholine or 1,3-dimedone at temperatures between 0 and 100° C., preferably at ambient temperature and under inert gas, or by treating with a catalytic amount of tris-(triphenylphosphine)-rhodium(I)chloride in a solvent such as aqueous ethanol and optionally in the presence of a base such as 1,4-diazabicyclo[2,2,2]octane at temperatures between 20 and 70° C.

The compounds of general formulae II to V used as starting materials, some of which are known from the literature, are obtained by methods known from the literature and their preparation is also described in the Examples.

The chemistry of the compounds of general formula II to V is described, for example, by Schröter in Stickstoffverbindungen II, pages 341-730, Methoden der organischen Chemie (Houben-Weyl), 4^{th} edition, Verlag Thieme, Stuttgart 1957. The preparation of carboxylic acid derivatives of general formula III is described in Methoden der organischen Chemie (Houben-Weyl), Volume E5, Carbonsäuren und Carbonsäurederivate, 4^{th} edition, Verlag Thieme, Stuttgart 1985.

Moreover, the compounds of general formula I obtained may be resolved into their enantiomers and/or diastereomers.

Thus, for example, the compounds of general formula I obtained which occur as racemates may be separated by methods known per se (cf. Allinger N. L. and Eliel E. L. in “Topics in Stereochemistry”, Vol. 6, Wiley Interscience, 1971) into their optical enantiomers and compounds of general formula I with at least 2 asymmetric carbon atoms may be resolved into their diastereomers on the basis of their physical-chemical differences using methods known per se, e.g. by chromatography and/or fractional crystallisation, and, if these compounds are obtained in racemic form, they may subsequently be resolved into the enantiomers as mentioned above.

The enantiomers are preferably separated by column separation on chiral phases or by recrystallisation from an optically active solvent or by reacting with an optically active substance which forms salts or derivatives such as e.g. esters or amides with the racemic compound, particularly acids and the activated derivatives or alcohols thereof, and separating the diastereomeric mixture of salts or derivatives thus obtained, e.g. on the basis of their differences in solubility, whilst the free antipodes may be released from the pure diastereomeric salts or derivatives by the action of suitable agents. Optically active acids in common use are e.g. the D- and L-forms of tartaric acid or dibenzoyltartaric acid, di-o-tolyltartaric acid, malic acid, mandelic acid, camphorsulphonic acid, glutamic acid, aspartic acid or quinic acid. An optically active alcohol may be for example (+) or (−)-menthol and an optically active acyl group in amides, for example, may be a (+)- or (−)-menthyloxycarbonyl.

Furthermore, the compounds of formula I may be converted into the salts thereof, particularly for pharmaceutical use into the physiologically acceptable salts with inorganic or organic acids. Acids which may be used for this purpose include for example hydrochloric acid, hydrobromic acid, sulphuric acid, methanesulphonic acid, phosphoric acid, fumaric acid, succinic acid, lactic acid, citric acid, tartaric acid or maleic acid.

Moreover, if the new compounds of formula I contain a carboxy group, they may subsequently, if desired, be converted into the salts thereof with inorganic or organic bases, particularly for pharmaceutical use into the physiologically acceptable salts thereof. Suitable bases for this purpose include for example sodium hydroxide, potassium hydroxide, cyclohexylamine, ethanolamine, diethanolamine and triethanolamine.

As already mentioned, the new compounds of general formula I and the salts thereof have valuable properties. Thus, the compounds of general formula I wherein Ar denotes a phenyl or naphthyl group substituted by the groups R₅, R₆ and R₇ and R₅ denotes a cyano group are valuable intermediates for preparing the corresponding compounds of general formula I wherein R₅ denotes an amidino group optionally substituted by one or two C₁₋₃-alkyl groups. The compounds of general formula I with the exception of those compounds wherein Ar denotes a phenyl or naphthyl group substituted by the groups R₅, R₆ and R₇ and R₅ denotes a cyano group, as well as the tautomers, the stereoisomers and the physiologically acceptable salts thereof, have valuable pharmacological properties, particularly an antithrombotic activity which is preferably based on an effect on thrombin or factor Xa, for example on a thrombin-inhibiting or factor Xa-inhibiting activity, on a prolonging effect on aPTT time and on an inhibitory effect on related serine proteases such as e.g. trypsin, urokinase, factor VIIa, factor IX, factor XI and factor XII.

For example, the compounds

(1) 2-(5-amidino-2-hydroxy-phenyl)-N-[3-methyl-4-((2-pyrrolidin-1-yl)-benzimidazol-1yl)-phenyl]-acetamide

(2) 2-(5-amidino-2-hydroxy-phenyl)-N-[3-methyl-4-(4,5,6,7-tetrahydro-benzimidazol-1-yl)-phenyl]-acetamide

(3) 2-(5-amidino-2-hydroxy-phenyl)-N-[4-(6,7-dihydro-5H-cyclopentapyrimidin-4-yl)-3-methyl-phenyl]-acetamide

(4) N-(5-amidino-2-hydroxy-benzyl)-3-trifluoromethyl-4-(3-methyl-1,4,5,6-tetrahydro-cyclopentapyrazol-1-yl)-benzamide

(5) N-(5-amidino-2-hydroxy-benzyl)-3-trifluoromethyl-4-(3-methylaminocarbonyl-1,4,5,6-tetrahydro-cyclopentapyrazol-1-yl)-benzamide

(6) N-(5-amidino-2-hydroxy-benzyl)-3-trifluoromethyl-4-(3-aminomethyl-1,4,5,6-tetrahydro-cyclopentapyrazol-1-yl)-benzamide

(7) ethyl 3-(3-amidino-phenyl)-3-[3-trifluoromethyl-4-(3-aminomethyl-1,4,5,6-tetrahydro-cyclopentapyrazol-1-yl)-benzoylamino]-propionate

(8) ethyl 3-(3-amidino-phenyl)-3-[3-trifluoromethyl-4-(4,5,6,7-tetrahydro-benzimidazol-1-yl)-benzoylamino]-propionate

(9) N-(5-amidino-2-hydroxy-benzyl)-3-trifluoromethyl-4-(3-methoxycarbonyl-1,4,5,6-tetrahydro-cyclopentapyrazol-1-yl)-benzamide

were investigated for their effect on the inhibition of factor Xa as follows:

Method: Enzyme-kinetic measurement with chromogenic substrate. The quantity of p-nitroaniline (pNA) released from the colourless chromogenic substrate by human factor Xa is determined photometrically at 405 nm. It is proportional to the activity of the enzyme used. The inhibition of the enzyme activity by the test substance (in relation to the solvent control) is determined at various concentrations of test substance and from this the IC₅₀ is calculated, as the concentration which inhibits the factor Xa used by 50%.

Material: Tris(hydroxymethyl)-aminomethane buffer (100 mmol) and sodium chloride (150 mmol), pH 8.0

Factor Xa (Roche), spec. activity: 10 U/0.5 ml, final concentration: 0.175 U/ml for each reaction mixture

Substrate Chromozym X (Roche), final concentration: 200 μMol/l for each reaction mixture

Test substance: final concentration 100, 30, 10, 3, 1, 0.3, 0.1, 0.03, 0.01, 0.003, 0.001 μMol/l

Procedure: 10 μl of a 23.5-times concentrated starting solution of the test substance or solvent (control), 175 μl of tris(hydroxymethyl)-aminomethane buffer and 25 μl of a 1.65 U/ml Factor Xa working solution are incubated for 10 minutes at 37° C. After the addition of 25 μl of Chromozym X working solution (1.88 μMol/l) the sample is measured in a photometer (SpectraMax 250) at 405 nm for 150 seconds at 37° C.

Evaluation

1. Determining the maximum increase (deltaOD/minutes) over 3 measuring points.

2. Determining the % inhibition based on the solvent control.

3. Plotting a dosage/activity curve (% inhibition vs substance concentration).

4. Determining the IC₅₀ by interpolating the X value (substance concentration) of the dosage/activity curve at Y=50% inhibition.

The following Table shows the results obtained:

| | Inhibition of factor Xa |
|---|---|
| Substance | (IC₅₀ in μM) |
| (1) | 0.14 |
| (2) | 0.081 |
| (3) | 0.062 |
| (4) | 0.015 |
| (5) | 0.016 |
| (6) | 0.007 |
| (7) | 0.0075 |
| (8) | 0.028 |
| (9) | 0.024 |

The compounds prepared according to the invention are well tolerated, as no toxic side effects could be observed at therapeutic doses.

In view of their pharmacological properties the new compounds, with the exception of those compounds wherein R⁴ denotes a cyano group, and the physiologically acceptable salts thereof are suitable for the prevention and treatment of venous and arterial thrombotic diseases, such as for example the treatment of deep leg vein thrombosis, for preventing reocclusions after bypass operations or angioplasty (PTCA), and occlusion in peripheral arterial diseases such as pulmonary embolism, disseminated intravascular coagulation, for preventing coronary thrombosis, stroke and the occlusion of shunts. In addition, the compounds according to the invention are suitable for antithrombotic support in thrombolytic treatment, such as for example with alteplase, reteplase, tenecteplase, staphylokinase or streptokinase, for preventing long-term restenosis after PTCA, for the prevention and treatment of ischaemic incidents in patients with unstable angina or non-transmural cardiac infarct, for preventing metastasis and the growth of clot-dependent tumours and fibrin-dependent inflammatory processes, e.g. in the treatment of pulmonary fibrosis, for preventing and treating rheumatoid arthritis, for preventing and treating fibrin-dependent tissue adhesions and/or the formation of scar tissue and for promoting wound healing processes. The new compounds and the physiologically acceptable salts thereof may be used therapeutically in conjunction with inhibitors of platelet aggregation such as fibrinogen receptor antagonists (e.g. abciximab, eptifibatide, tirofiban), with inhibitors of ADP-induced aggregation (e.g. clopidogrel, ticlopidine), with P₂T receptor antagonists (e.g. cangrelor) or with combined thromboxane receptor antagonists/synthetase inhibitors (e.g. terbogrel).

The dosage required to achieve such an effect is appropriately 3 to 30 mg/kg, preferably 1 to 10 mg/kg by intravenous route, and 5 to 50 mg/kg, preferably 3 to 30 mg/kg by oral route, in each case administered 1 to 4 times a day. For this purpose, the compounds of formula I prepared according to the invention may be formulated, optionally together with other active substances, with one or more inert conventional carriers and/or diluents, e.g. with corn starch, lactose, glucose, microcrystalline cellulose, magnesium stearate, polyvinylpyrrolidone, citric acid, tartaric acid, water, water/ethanol, water/glycerol, water/sorbitol, water/polyethylene glycol, propylene glycol, cetylstearyl alcohol, carboxymethylcellulose or fatty substances such as hard fat or suitable mixtures thereof, to produce conventional galenic preparations such as plain or coated tablets, capsules, powders, suspensions or suppositories.

The Examples which follow are intended to illustrate the invention:

### EXAMPLE 1

### 2-(5-amidino-2-hydroxy-phenyl)-N-[3-methyl-4-(2-tert.butoxycarbonylaminomethyl-benzimidazol-1-yl)-phenyl]-acetamide

a. tert.-butyl (1H-benzimidazol-2-yl-methyl)-carbaminate

5.5 g (25 mmol) of 2-aminomethyl-benzimidazole are suspended in 50 ml of dioxane and 20 ml of water. The clear solution obtained after the addition of 10.5 ml (75 mmol) of triethylamine is combined at ambient temperature with a solution of 5.5 g of di-tert. butyl pyrocarbonate in 50 ml of dioxane. After 1.5 hours the reaction mixture is diluted with ethyl acetate, and the aqueous phase is separated off. The organic phases are washed 2× with water and saturated sodium chloride solution, dried over magnesium sulphate and evaporated down.

Yield: 6.2 g (100% of theory) C₁₃H₁₇N₃O₂(247.30)

Mass spectrum: (M+H)⁺=248 (M−H)⁻=246

b. tert. butyl [1-(2-methyl-4-nitro-phenyl)-1H-benzimidazol-2-yl-methyl]-carbamidate

A solution of 2.5 g (10 mmol) of tert. butyl (1H-benzimidazol-2-yl-methyl)-carbaminate and 1.6 g (10 mmol) of 2-fluoro-5-nitro-toluene in 30 ml of dimethylformamide is combined with 0.5 g (10.8 mmol) of sodium hydride. The reaction solution is heated to 100° C. for 2 hours. After another 12 hours at ambient temperature the reaction solution is poured onto 200 ml of ice water. The yellow precipitate formed is extracted with ethyl acetate, washed with water and saturated saline solution, dried and evaporated down. The residue is triturated with ether, suction filtered and dried.

Yield: 0.7 g (17% of theory) C₂₀H₂₂N₄O₄ (382.42)

Mass spectrum: (M+H)⁺=383 (M−H)⁻=381

c. tert. butyl [1-(2-methyl-4-amino-phenyl)-1H-benzimidazol-2-yl-methyl]-carbamidate 0.6 g (1.6 mmol) of tert. butyl [1-(2-methyl-4-nitro-phenyl)-1H-benzimidazol-2-yl -methyl]-carbamidate are dissolved in 70 ml of methanol and, after the addition of 0.2 g of palladium on activated charcoal/hydrogen (20%), hydrogenated with hydrogen for 1.5 hours at ambient temperature. Then the catalyst is filtered off and the filtrate is evaporated down.

Yield: 0.55 g (99% of theory),

R_{f} value: 0.45 (silica gel; dichloromethane/ethanol=9:1+ammonia) C₂₀H₂₄N₄O₂ (352.44)

Mass spectrum: (M+H)⁺=353 (M−H)⁻=351

d. 4-Allyloxy-benzonitrile

100.1 g (0.84 mol) of 4-hydroxy-benzonitrile are dissolved in 600 ml of dimethylformamide and after the addition of 103.2 g (0.92 mol) of potassium tert. butoxide stirred for 30 minutes at 35° C. Then a solution of 79.6 ml (0.92 mol) of 3-bromo-propene in 10 ml of dimethylformamide is added dropwise. After 1 hour at 60° C. the reaction solution is poured onto ice water, combined with 300 ml of 2 molar sodium hydroxide solution and extracted with ethyl acetate. The organic extracts are dried and evaporated down.

Yield: 130.1 g (97% of theory),

R_{f} value: 0.25 (silica gel; petroleum ether/ethyl acetate=8:2)

e. 3-allyl-4-hydroxy-benzonitrile

47.8 g (0.3 mol) of 4-allyloxy-benzonitrile are heated to 210° C. under a nitrogen atmosphere for 60 minutes. After cooling the residue is chromatographed on silica gel, eluting with petroleum ether/ethyl acetate (9:1 and 1:1).

Yield: 14.7 g (31% of theory),

R_{f} value: 0.45 (silica gel; petroleum ether/ethyl acetate=1:1)

f. 3-allyl-4-benzyloxy-benzonitrile

14.6 g (0.09 mol) of 3-allyl-4-hydroxy-benzonitrile and 34.6 g (0.25 mol) of potassium carbonate are stirred in 200 ml of dimethylformamide for 15 minutes. After the addition of 12.1 ml (0.1 mol) of benzylbromide the reaction mixture is stirred for 2 hours at ambient temperature. The reaction solution is poured onto ice water and the crystalline product is suction filtered.

Yield: 19.9 g (87% of theory),

R_{f} value: 0.55 (silica gel; petroleum ether/ethyl acetate=8:2)

g. (2-benzyloxy-5-cyano)-phenylacetic acid

5.0 g (20 mmol) of 3-allyl-4-benzyloxy-benzonitrile are added batchwise to a solution of 17.4 g (110 mmol) of potassium permanganate in 50 ml of water and 150 ml of glacial acetic acid, whereupon the temperature rises to 55° C. After 1 hour at 40° C. the reaction mixture is diluted with ethyl acetate and suction filtered through Celite. The organic phase is separated off and evaporated down. The residue is chromatographed on silica gel, eluting with petroleum ether/ethyl acetate (9:1 and 1:1).

Yield: 2.2 g (40% of theory),

R_{f} value: 0.2 (silica gel; petroleum ether/ethyl acetate 1:1)

h. 2-(5-cyano-2-benzyloxy-phenyl)-N-[3-methyl-4-(2-tert.butoxycarbonylaminomethyl-benzimidazol-1-yl)-phenyl]-acetamide

A solution of 0.6 g (1.7 mmol) of tert. butyl [1-(2-methyl-4-amino-phenyl)-1H -benzimidazol-2-yl-methyl]-carbamidate and 0.45 g (1.7 mmol) of (2-benzyloxy-5-cyano)-phenylacetic acid in 10 ml of dimethylformamide is combined with 0.2 ml (1.8 mmol) of N-methylmorpholine and 0.58 g (1.8 mmol) of O-(benzotriazol-1-yl)-N,N,N′,N′-tetramethyluronium tetrafluoroborate and stirred for 5 hours at ambient temperature. The reaction mixture is combined with 200 ml of ice water, the precipitate formed is suction filtered, taken up in ethyl acetate, extracted with water and saturated saline solution and dried. The crude product is purified on silica gel, eluting with dichloromethane and continuously increasing amounts of ethyl acetate (0-30%). The uniform fractions are combined and evaporated down.

Yield: 0.65 g (64% of theory),

R_{f} value: 0.32 (silica gel, dichloromethane/ethyl acetate=7:3)

i. 2-(5-N′-hydroxy-amidino-2-benzyloxy-phenyl)-N-[3-methyl-4-(2-tert.butoxycarbonylaminomethyl-benzimidazol-1-yl)-phenyl-acetamide

A solution of 0.6 g (1.06 mmol) of 2-(5-cyano-2-benzyloxy-phenyl)-N-[3-methyl-4-(2-tert.butoxycarbonylaminomethyl-benzimidazol-1-yl)-phenyl]-acetamide in 20 ml of ethanol/methanol (1:1) is combined with a solution of 0.2 g (2.1 mmol) of sodium acetate in 0.25 ml of water. After the addition of a solution of 0.1 g (2.1 mmol) of hydroxylamine hydrochloride in 0.35 ml of water the reaction mixture is refluxed for 2 hours. After cooling the crude product is purified on silica gel, eluting with ethyl acetate/methanol plus 0-1% ammonia (5%). The uniform fractions are combined and evaporated down.

Yield: 0.6 g (89% of theory)

R_{f} value: 0.63 (silica gel, ethyl acetate/ethanol 9:1+ammonia)

k. 2-(5-amidino-2-hydroxy-phenyl)-N-[3-methyl-4-(2-tert.butoxycarbonylaminomethyl -benzimidazol-1-yl)-phenyl]-acetamide

Prepared analogously to Example 1.c. from 2-(5-N′-hydroxy-amidino-2-benzyloxy -phenyl)-N-[3-methyl-4-(2-tert.butoxycarbonylaminomethyl-benzimidazol-1-yl)-phenyl]-acetamide and palladium on activated charcoal (10%)/hydrogen in methanol/glacial acetic acid.

Yield: 30% of theory, C₂₉H₃₂N₆O₄ (528.61)

Mass spectrum: (M+H)⁺=529 (M−H)⁻=527

### EXAMPLE 2

### N-(5-amidino-2-hydroxy-phenyl)-3-methyl-4-(2-aminomethyl-benzimidazol-1-yl)-phenyl-acetamide-dihydrochloride

0.1 g (0.19 mmol) of N-(5-amidino-2-hydroxy-phenyl)-3-methyl-4-(2-tert.butoxycarbonylaminomethyl-benzimidazol-1-yl)-phenyl-acetamide are dissolved in 10 ml of ethanolic hydrochloric acid and stirred for 15 minutes at 40° C. The solution is evaporated down, taken up in a little ethanol, overlaid with ether and stirred for 30 minutes. The solid formed is suction filtered and dried.

Yield: 0.04 g (42% of theory),

R_{f} value: 0.38 (silica gel; dichloromethane/methanol/ammonia=4:1:0.1) C₂₄H₂₄N₆O₂×2HCl (428.50/501.42)

Mass spectrum: (M+H)⁺=429 (M−H)⁻=427

The following compounds are obtained analogously to Example 2:

### (1) N-(5-amidino-2-hydroxy-benzyl)-3-trifluoromethyl-4-(3-aminomethyl-1,4,5,6-tetrahydro-cyclopentapyrazol-1-yl)-benzamide-bistrifluoroacetate

Yield: 74% of theory,

R_{f} value: 0.69 (Reversed phase RP 8; 5% sodium chloride solution/methanol 1:3) C₂₃H₂₃F₃N₆O₂ (472.47/700.52)

Mass spectrum: (M+H)⁺=473 (M−H)⁻=471

### (2) ethyl 3-(3-amidino-phenyl)-3-[13-trifluoromethyl-4-(3-aminomethyl-1,4,5,6-tetrahydro-cyclopentapyrazol-1-yl)-benzoylamino]-propionate bistrifluoroacetate

Yield: 78% of theory,

R_{f} value: 0.64 (Reversed phase RP 8; 5% sodium chloride solution/methanol=1:3) C₂₇H₂₉F₃N₆O₃ (542.57/770.62)

Mass spectrum: (M−H)⁻=541 (M+Na)⁺=565

### (3) ethyl 3-(3-aminomethyl-phenyl)-3-[3-trifluoromethyl-4-(3-aminomethyl-1,4,5,6-tetrahydro-cyclopentapyrazol-1-yl)-benzoylamino]-propionate

Yield: 56% of theory,

R_{f} value: 0.45 (silica gel; dichloromethane/methanol 7:1+1% ammonia) C₂₇H₃₀F₃N₅O₃ (529.572)

Mass spectrum: (M+H)⁺=530 M−H)⁻=528

### EXAMPLE 3

### 2-(5-amidino-2-hydroxy-phenyl)-N-[4-(4,5-dimethyl-2-oxo-2,3-dihydroimidazol-1-yl) -3-methyl-phenyl]-acetamide-hydrochloride

a. 4.5-Dimethyl-3H-oxazol-2-one

50 g (0.56 mol) of acetone are dissolved in 225 ml of dimethylformamide and after the addition of 151.4 g (1.7 mol) of ethylcarbamate the mixture is refluxed for 29 hours under a nitrogen atmosphere. The solvent is distilled off and the residue is recrystallised from ethanol/petroleum ether (1:2).

Yield: 6.1 g (9.5% of theory),

R_{f} value: 0.56 (silica gel, ethyl acetate)

Melting point: 106-108° C.

b. 4.5-Dimethyl-2-oxo-oxazol-3-carboxylic acid-(2-methyl-4-nitro-phenyl)-amide

6 g (53 mmol) of 4,5-dimethyl-3H-oxazol-2-one are dissolved in 30 ml of dimethylformamide and after the addition of 11.2 g (63 mmol) of 2-methyl-4-nitrophenyl isocyanate the mixture is stirred for 2 hours at 35° C. Then the reaction mixture is stirred with 400 ml of ice water and suction filtered. The residue is recrystallised from 500 ml of ethanol.

Yield: 10.9 g (59% of theory),

R_{f} value: 0.53 (silica gel, cyclohexane/ethyl acetate 2:1)

c. 4,5-Dimethyl-1-(2-methyl-4-nitro-phenyl)-1,3-dihydro-imidazol-2-one

9.4 g (32 mmol) of 4,5-dimethyl-2-oxo-oxazol-3-carboxylic acid-(2-methyl-4-nitro -phenyl)-amide are stirred in 250 ml of glacial acetic acid and 35 ml (309 mmol) of hydrobromic acid (48%) for 1 hour at 120° C. Then the mixture is evaporated down by 80%, the residue is stirred with ice water and suction filtered. The crude product is chromatographed on silica gel, eluting with dichloromethane/methanol (50:1, 25:1 and 9:1).

Yield: 3.3 g (41% of theory),

R_{f} value: 0.54 (silica gel, ethyl acetate/ethanol 9:1)

d. 1-(4-amino-2-methyl-phenyl)-4,5-dimethyl-1,3-dihydroimidazol-2-one

Prepared analogously to Example 1.c. from 4,5-dimethyl-1-(2-methyl-4-nitro-phenyl)-1,3-dihydro-imidazol-2-one and palladium on activated charcoal/hydrogen in methanol.

Yield: 93% of theory,

R_{f} value: 0.3 (silica gel, ethyl acetate/toluene/ammonia=9:0.9:0.1)

e. 2-(2-benzyloxy-5-cyano-phenyl)-N-[4-(4,5-dimethyl-2-oxo-2,3-dihydroimidazol-1-yl) -3-methyl-phenyl]-acetamide

Prepared analogously to Example 1.h. from 1-(4-amino-2-methyl-phenyl)-4,5-dimethyl -1,3-dihydroimidazol-2-one, (2-benzyloxy-5-cyano)-phenylacetic acid, O-(benzotriazol-1-yl)-N,N,N′,N′-tetramethyluronium tetrafluoroborate and N-methylmorpholine in dimethylformamide

Yield: 80% of theory,

R_{f} value: 0.6 (silica gel, ethyl acetate/ethanol=9:1)

f. 2-(5-amidino-2-benzyloxy-phenyl)-N-[4-(4,5-dimethyl-2-oxo-2,3-dihydroimidazol-1-yl)-3-methyl-phenyl]-acetamide-hydrochloride

1.5 g (3.2 mmol) of 2-(2-benzyloxy-5-cyano-phenyl)-N-[4-(4,5-dimethyl-2-oxo-2,3-dihydroimidazol-1-yl)-3-methyl-phenyl]-acetamide are dissolved in 65 ml of saturated ethanolic hydrochloric acid and stirred for 23 hours at ambient temperature. The solvent is distilled off, the residue is dissolved in 20 ml of absolute ethanol and combined with 4.6 g (48 mmol) of ammonium carbonate. After 16 hours at ambient temperature it is evaporated to dryness. The residue is stirred with water, suction filtered and dried.

Yield: 1.1 g (66% of theory),

R_{f} value: 0.55 (Reversed phase RP 8; 5% sodium chloride solution/methanol=1:3)

g. 2-(5-amidino-2-hydroxy-phenyl)-N-[4-(4.5-dimethyl-2-oxo-2.3-dihydroimidazol-1-yl) -3-methyl-phenyl]-acetamide-hydrochloride

Prepared analogously to Example 1.c. from 2-(5-amidino-2-benzyloxy-phenyl)-N-[4-(4,5-dimethyl-2-oxo-2,3-dihydroimidazol-1-yl)-3-methyl-phenyl]-acetamide-hydrochloride and hydrogen/palladium on activated charcoal in methanol.

Yield: 48% of theory,

R_{f} value: 0.67 (Reversed phase RP 8; 5% sodium chloride solution/methanol=1:2) C₂₁H₂₃N₅O₃×HCl (393.44/429.91)

Mass spectrum: (M+H)⁺=394 (M−H)⁻=392

### EXAMPLE 4

### 2-(5-amidino-2-hydroxy-phenyl)-N-[4-(benzimidazol-1-yl)-3-methyl-phenyl]-acetamide-hydrochloride

a. 4-(benzimidazol-1-yl)-3-methyl-nitrobenzene

0.6 g (5 mmol) of benzimidazole are dissolved in 15 ml of tetrahydrofuran and after the addition of 0.2 g (5 mmol) of sodium hydride stirred for 30 minutes. After the addition of 0.8 g (5 mmol) of 4-fluoro-3-methyl-nitrobenzene the reaction mixture is stirred for 2.5 hours at 65° C. After cooling, 100 ml of ice water are added and the product precipitated is suction filtered and dried.

Yield: 1.2 g (91% of theory),

R_{f} value: 0.59 (silica gel; dichloromethane/ethanol 19:1)

b. 4-(benzimidazol-1-yl)-3-methyl-aniline

Prepared analogously to Example 1.c. from 4-(benzimidazol-1-yl)-3-methyl-nitrobenzene and palladium on activated charcoal in methanol.

Yield: 98% of theory,

R_{f} value: 0.5 (silica gel; dichloromethane/ethanol/ammonia 19:1:0.1)

c. (2-benzyloxy-5-cyano)-phenylacetic acid chloride

1.2 g (4.5 mmol) of (2-benzyloxy-5-cyano)-phenylacetic acid,1ml (13.7 mmol) of thionylchloride, 0.01 ml of dimethylformamide and 30 ml of dichloromethane are refluxed for 1.25 h. Then the solvent is distilled off and the crude product is reacted without purification.

Yield: 4.2 g (100% of theory),

d. 2-(2-benzyloxy-5-cyano-phenyl)-N-[4-(benzimidazol-1-yl)-3-methyl-phenyl]-acetamide

While cooling with ice, a solution of 1.3 g (4.5 mmol) of (2-benzyloxy-5-cyanophenyl)-acetic acid chloride in 30 ml of tetrahydrofuran is added dropwise within 20 minutes to a solution of1 g (4.2 mmol) of 4-(benzimidazol-1-yl)-3-methyl-aniline and 1.9 ml (13.5 mmol) of triethylamine in 15 ml of tetrahydrofuran. The reaction solution is stirred for 16 hours at ambient temperature, mixed with ice water and suction filtered. The crude product is chromatographed on silica gel, eluting with dichloromethane/ethyl acetate (1:0 and 1:1).

Yield: 1.1 g (55% of theory),

R_{f} value: 0.55 (silica gel; ethyl acetate+1drop of ammonia)

e. 2-(5-amidino-2-hydroxy-phenyl)-N-[4-(benzimidazol-1-yl)-3-methyl-phenyl]-acetamide-hydrochloride

Prepared analogously to Example 3.f. from 2-(2-benzyloxy-5-cyano-phenyl)-N-[4-(benzimidazol-1-yl)-3-methyl-phenyl]-acetamide and hydrochloric acid/ammonium carbonate in ethanol followed by reaction with hydrogen/palladium on activated charcoal in methanol analogously to Example 1.c.

Yield: 53% of theory,

R_{f} value: 0.78 (Reversed phase RP 8; 5% sodium chloride solution/methanol=1:3) C₂₃H₂₁N₅O₂×HCl (399.45/435.91)

Mass spectrum: (M+H)⁺=400 (M−H)⁻=398 (M+Cl)⁻=434/36 (Cl)

The following compounds are prepared analogously to Example 4:

### (1) 2-(5-amidino-2-hydroxy-phenyl)-N-[3-methyl-4-(2-methyl-benzimidazol-1-yl) -phenyl]-acetamide-hydrochloride

Yield: 78% of theory,

R_{f} value: 0.55 (Reversed phase RP 8; 5% sodium chloride solution/methanol=1:1) C₂₄H₂₃N₅O₂×HCl (413,48/449.94)

Mass spectrum: (M+H)⁺=414 (M−H)⁻=412 (M+Cl)⁻=448/50 (Cl)

### (2) 2-(5-amidino-2-hydroxy-phenyl)-N-[3-methyl-4-((2-pyrrolidin-1-yl)-benzimidazol -1-yl)-phenyl]-acetamide-hydrochloride

Yield: 97% of theory,

R_{f} value: 0.71 (Reversed phase RP 8; 5% sodium chloride solution/methanol 1:3) C₂₇H₂₈N₆O₂×HCl (468.56/505.02)

Mass spectrum: (M+H)⁺=469 (M−H)⁻=467 (M+Cl)⁻=503/05 (Cl)

### (3) 2-(5-amidino-2-hydroxy-phenyl)-N-[3-methyl-4-(2-dimethylamino-benzimidazol -1-yl)-phenyl]-acetamide-hydrochloride

Yield: 40% of theory,

R_{f} value: 0.65 (Reversed phase RP 8; 5% sodium chloride solution/methanol=2:3) C₂₅H₂₆N₆O₂×HCl (442.52/478.99)

Mass spectrum: (M+H)⁺=443 (M−H)⁻=441 (M+Cl)⁻=477 (Cl)

### (4) 2-(5-amidino-2-hydroxy-phenyl)-N-[3-methyl-4-(4,5,6,7-tetrahydro-benzimidazol-1-yl)-phenyl]-acetamide-hydrochloride

Yield: 12% of theory,

R_{f} value: 0.43 (Reversed phase RP 8; 5% sodium chloride solution/methanol=2:3) C₂₃H₂₅N₅O₂×HCl (403.49/439.95)

Mass spectrum: (M+H)⁺=404

### (5) N-(5-amidino-2-hydroxy-benzyl)-3-trifluoromethyl-4-(4,5,6,7-tetrahydro -benzimidazol-1-yl)-benzamide-hydrochloride

Yield: 57% of theory,

R_{f} value: 0.67 (Reversed phase RP 8; 5% sodium chloride solution/methanol=1:3) C₂₃H₂₂F₃N₅O₂×HCl (457.64/493.92)

Mass spectrum: (M+H)⁺=458 (M−H)⁻=456 (M+Cl)⁻=492/4 (Cl)

### (6) N-[1-(5-amidino-2-hydroxy-phenyl)-ethyl]-3-trifluoromethyl-4-(4,5,6,7-tetrahydro-benzimidazo-1-yl)-benzamide-hydrochloride

Yield: 47% of theory,

R_{f} value: 0.3 (Reversed phase RP 8; 5% sodium chloride solution/methanol=2:3) C₂₄H₂₄F₃N₅O₂×HCl (471.49/507.94)

Mass spectrum: (M+H)⁺=472 (M−H)⁻=470 (M+Cl)⁻=506/8 (Cl)

### (7) 2-(5-amidino-2-hydroxy-phenyl)-N-[3-trifluoromethyl-4-(4,5,6,7-tetrahydro-benzimidazol-1-yl)-phenyl]-propionamide-hydrochloride

Yield: 79% of theory,

R_{f} value: 0.5 (Reversed phase RP 8; 5% sodium chloride solution/methanol=2:3) C₂₄H₂₄F₃N₅O₂×HCl (471.49/507.94)

Mass spectrum: (M+H)⁺=472

### (8) 2-(5-amidino-2-hydroxy-phenyl)-N-[3-trifluoromethyl-4-(4,5,6,7-tetrahydro-benzimidazol-1-yl)-phenyl]-acetamide-hydrochloride

Yield: 76% of theory,

R_{f} value: 0.55 (Reversed phase RP 8; 5% sodium chloride solution/methanol=2:3) C₂₃H₂₂F₃N₅O₂×HCl (457.45/493.92)

Mass spectrum: (M+H)⁺=458

### (9) N-(5-amidino-2-hydroxy-benzyl)-3-trifluoromethyl-4-(5,6-dihydro-4H-cyclopentaimidazol-1-yl)-benzamide-hydrochloride

Yield: 74% of theory,

R_{f} value: 0.46 (Reversed phase RP 8; 5% sodium chloride solution/methanol=1:2) C₂₂H₂₀F₃N₅O₂×HCl (443.43/479.9)

Mass spectrum: (M+Cl)⁻=478/80 (Cl)

### (10) N-[1-(5-amidino-2-hydroxy-phenyl)-ethyl]-3-trifluoromethyl-4-(5,6-dihydro-41H-cyclopentaimidazol-1-yl)-benzamide-hydrochloride

Yield: 70% of theory,

R_{f} value: 0.56 (Reversed phase RP 8; 5% sodium chloride solution/methanol=1:2) C₂₃H₂₂F₃N₅O₂×HCl (457.45/493.92)

Mass spectrum: (M+H)⁺=458 (M−H)⁻=456 (M+Cl)⁻=492

### EXAMPLE 5

### 2-(5-amidino-2-hydroxy-phenyl)-N-[3-methyl-4-(3-ethoxycarbonyl-1,4,5,6-tetrahydro -cyclopentapyrazol-1-yl)-phenyl]-acetamide-hydrochloride

a. (2-methyl-4-nitro-phenyl)-hydrazine

4.7 g (30 mmol) of 2-fluoro-5-nitrotoluene and 5 ml (128 mmol) of hydrazine hydrate are refluxed for 5 hours in 50 ml of ethanol. After another 20 hours at ambient temperature the product is suction filtered, washed with ether and dried.

Yield: 2.7 g (54% of theory),

R_{f} value: 0.35 (silica gel; dichloromethane)

b. ethyl 1-(2-methyl-4-nitro-phenyl)-1,4,5,6-tetrahydro-cyclopentapyrazol-3-carboxylate 2.9 g (15.5 mmol) of ethyl 2-oxo-(2-oxo-cyclopentyl)-acetate and 2.6 g (15.5 mmol) of (2-methyl-4-nitro-phenyl)-hydrazine are refluxed in 50 ml of ethanol for 30 minutes. The reaction solution is cooled, the precipitate is suction filtered and washed with ethanol. The crude product is purified on silica gel, eluting with dichloromethane/methanol/ammonia (19:1:0.1).

Yield: 1.0 g (20% of theory),

R_{f} value: 0.29 (silica gel; dichloromethane)

c. ethyl 1-(2-methyl-4-amino-phenyl)-1,4,5,6-tetrahydro-cyclopentapyrazol-3-carboxylate

Prepared analogously to Example 1.c. from ethyl 1-(2-methyl-4-nitro-phenyl)-1,4,5,6-tetrahydro-cyclopentapyrazole-3-carboxylate and palladium on activated charcoal/hydrogen in methanol.

Yield: 100% of theory,

R_{f} value: 0.17 (silica gel; dichloromethane)

d. 2-(5-cyano-2-benzyloxy-phenyl)-N-[3-methyl-4-(3-ethoxycarbonyl-1,4,5,6-tetrahydro -cyclopentapyrazol-1-yl)-phenyl]-acetamide

Prepared analogously to Example 1.h. from ethyl 1-(2-methyl-4-amino-phenyl)-1,4,5,6-tetrahydro-cyclopentapyrazol-3-carboxylate and (2-benzyloxy-5-cyanophenyl)-acetic acid.

Yield: 52% of theory,

e. 2-(5-amidino-2-hydroxy-phenyl)-N-[3-methyl-4-(3-ethoxycarbonyl-1,4,5,6-tetrahydro -cyclopentapyrazol-1-yl)-phenyl]-acetamide-hydrochloride

Prepared analogously to Example 3.f. from 2-(5-cyano-2-benzyloxy-phenyl)-N-[3-methyl-4-(3-ethoxycarbonyl-1,4,5,6-tetrahydro-cyclopentapyrazol-1-yl)-phenyl]-acetamide and hydrochloric acid/ammonium carbonate in ethanol followed by reaction analogously to Example 1.c. in methanol with the addition of palladium on activated charcoal/hydrogen.

Yield: 28% of theory,

R_{f} value: 0.49 (silica gel; dichloromethane/methanol/ammonia 4:1:0.1) C₂₅H₂₇N₅O₄×HCl (461.53/497.98)

Mass spectrum: (M+H)⁺=462 (M−H)⁻=460 (M+Cl)⁻=496

The following compounds are prepared analogously to Example 5:

### (1) 2-(5-amidino-2-hydroxy-phenyl)-N-14-(3-methyl-1,4,5,6-tetrahydro-cyclopentapyrazol-1-yl)-phenyl]-acetamide-hydrochloride

Yield: 35% of theory,

R_{f} value: 0.48 (Reversed phase RP 8; 5% sodium chloride solution/methanol=1:3) C₂₂H₂₃N₅O₂×HCl (389.46/425.92)

Mass spectrum: (M+H)⁺=390

### (2) N-(5-amidino-2-hydroxy-benzyl)-3-methyl-4-(3-methyl-1,4,5,6-tetrahydro-cyclopentapyrazol-1-yl) -benzamide-hydrochloride

Yield: 88% of theory,

R_{f} value: 0.35 (Reversed phase RP 8; 5% sodium chloride solution/methanol=2:3) C₂₃H₂₅N₅O₂×HCl (403.49/439.95)

Mass spectrum: (M+H)⁺=404 (M−H)⁻=402 (M+Cl)⁻=438/40 (Cl)

### (3) 2-(5-amidino-2-hydroxy-phenyl)-N-[3-methyl-4-(3-methyl-1,4,5,6-tetrahydro-cyclopentapyrazol-1-yl)-phenyl]-acetamide-hydrochloride

Yield: 86% of theory,

R_{f} value: 0.55 (Reversed phase RP 8; 5% sodium chloride solution/methanol=1:2) C₂₃H₂₅N₅O₂×HCl (403.49/439.95)

Mass spectrum: (M+H)⁺=404 (M−H)⁻=402 (M+Cl)⁻=438

### EXAMPLE 6

### 2-(5-amidino-2-hydroxy-phenyl)-N-[3-methyl-4-(1-methyl-1,4,5,6-tetrahydro-cyclopentapyrazol-3-yl)-phenyl]-acetamide-hydrochloride

a. 2-(2-methyl-4-nitro-benzoyl)-cyclopentanone

A solution of 6.7 g (33.6 mmol) of 2-methyl-4-nitro-benzoic acid chloride in 15 ml of chloroform is added dropwise at −15° C. to a solution of 5.6 ml (33.6 mmol) of 1-morpholino-1-cyclopentene in 15 ml of chloroform. After 15 hours' stirring at ambient temperature and the addition of 10 ml of hydrochloric acid (4N) the reaction mixture is refluxed for 2.5 h. After cooling the organic phase is separated off, the aqueous phase is made alkaline with conc. ammonia and extracted with dichloromethane. The combined organic extracts are evaporated down and chromatographed on silica gel, eluting with petroleum ether/ethyl acetate (0-30%).

Yield: 4.6 g (55% of theory),

R_{f} value: 0.75 (silica gel; petroleum ether/ethyl acetate=1:1+glacial acetic acid)

b. 1-methyl-3-(2-methyl-4-nitro-phenyl)-1,4,5 6-tetrahydro-cyclopentapyrazole and 2-methyl-3-(2-methyl-4-nitro-phenyl)-2,4,5 6-tetrahydro-cyclopentapyrazole

0.6 g (2.5 mmol) of 2-(2-methyl-4-nitro-benzoyl)-cyclopentanone is added to a mixture of 1 ml (18.4 mmol) of methylhydrazine and 2 ml of glacial acetic acid at 20-30° C. After 1.5 hours at 100° C. the reaction solution is cooled, the precipitate formed is suction filtered and distributed in ethyl acetate/water. After the extraction the combined organic extracts are evaporated down and chromatographed on silica gel, eluting with petroleum ether/ethyl acetate (0-50%). Two products are isolated:

1. 1-methyl-3-(2-methyl-4-nitro-phenyl)-1,4,5,6-tetrahydro-cyclopentapyrazole

Yield: 0.3 g (44% of theory),

R_{f} value: 0.65 (silica gel; petroleum ether/ethyl acetate=1:1) C₁₄H₁₅N₃O₂ (257.29)

Mass spectrum: (M+H)⁺=258

2. 2-methyl-3-(2-methyl-4-nitro-phenyl)-2,4,5,6-tetrahydro-cyclopentapyrazole

Yield: 0.3 g (39% of theory),

R_{f} value: 0.49 (silica gel; petroleum ether/ethyl acetate=1:1) C₁₄H₁₅N₃O₂ (257.29)

Mass spectrum: (M+H)⁺=258

c. 1-methyl-3-(2-methyl-4-amino-phenyl)-1,4,5,6-tetrahydro-cyclopentapyrazole

Prepared analogously to Example 1.c. from 1-methyl-3-(2-methyl-4-nitro-phenyl)-1,4,5,6-tetrahydro-cyclopentapyrazole and palladium on activated charcoal/hydrogen (20%) in methanol.

Yield: 91% of theory,

R_{f} value: 0.27 (silica gel; petroleum ether/ethyl acetate=1:1) C₁₄H₁₇N₃ (227.31)

Mass spectrum: (M+H)⁺=228

d. 2-(5-cyano-2-benzyloxy-phenyl)-N-[3-methyl-4-(1-methyl-1,4,5,6-tetrahydro-cyclopentapyrazol-3-yl)-phenyl]-acetamide

Prepared analogously to Example 1.h. from 1-methyl-3-(2-methyl-4-amino-phenyl) -1,4,5,6-tetrahydro-cyclopentapyrazole, (2-benzyloxy-5-cyanophenyl)-acetic acid, O-(benzotriazol-1-yl)-N,N,N′-N′-tetramethyluronium tetrafluoroborate and N-methylmorpholine in dimethylformamide.

Yield: 95% of theory,

R_{f} value: 0.83 (silica gel; ethyl acetate+ammonia)

e. 2-(5-amidino-2-hydroxy-phenyl)-N-[3-methyl-4-(1-methyl-1,4,5,6-tetrahydro-cyclopentapyrazol-3-yl)-phenyl]-acetamide-hydrochloride

Prepared analogously to Example 3.f. from 2-(5-cyano-2-benzyloxy-phenyl)-N-[3-methyl-4-(1-methyl-1,4,5,6-tetrahydro-cyclopentapyrazol-3-yl)-phenyl]-acetamide and hydrochloric acid/ammonium carbonate in ethanol followed by reaction with hydrogen/palladium on activated charcoal in methanol analogously to Example 1.c.

Yield: 54% of theory,

R_{f} value: 0.33 (Reversed phase RP 8; 5% sodium chloride solution/methanol=1:3) C₂₃H₂₅N₅O₂×HCl (403.49/439.95)

Mass spectrum: (M+H)⁺=404 (M−H)⁻=402 (M+Cl)⁻=438/40 (Cl)

The following compounds are prepared analogously to Example 6:

### (1) 2-(5-amidino-2-hydroxy-phenyl)-N-[3-methyl-4-(2-methyl-2,4,5,6-tetrahydro-cyclopentapyrazol-3-yl)-phenyl]-acetamide-hydrochloride

Yield: 100% of theory, C₂₃H₂₅N₅O₂×HCl (403.49/439.95)

Mass spectrum: (M+H)⁺=404 (M−H)⁻=402 (M+Cl)⁻=438/40 (Cl)

### (2) 2-(5-amidino-2-hydroxy-phenyl)-N-[3-methyl-4-(1,4,5,6-tetrahydro-cyclopentapyrazol-3-yl)-phenyl]-acetamide-hydrochloride

Yield: 56% of theory, C₂₂H₂₃N₅O₂×HCl (389.46/425.95)

Mass spectrum: (M+H)⁺=390 (M−H)⁻=388 (M+Cl)⁻=424 (Cl)

### EXAMPLE 7

### 2-(5-amidino-2-hydroxy-phenyl)-N-[4-(3-dimethylaminomethyl-1,4,5,6-tetrahydro-cyclopentapyrazol-1-yl)-3-methyl-phenyl]-acetamide-hydrochloride

a. [1-(2-methyl-4-nitro-phenyl)-1,4,5,6-tetrahydro-cyclopentapyrazol-3-yl]-methanol

1.6 g (5.4 mmol) of methyl 1-(2-methyl-4-nitro-phenyl)-1,4,5,6-tetrahydro-cyclopentapyrazol-3-carboxylate are dissolved in 130 ml of dioxane, combined with 80 ml of water and after the addition of 2 g (52.8 mmol) of sodium borohydride stirred for 40 hours at ambient temperature. The mixture is then combined with ice, acidified with 2 molar hydrochloric acid and extracted with ethyl acetate. The organic phase is evaporated down and purified on silica gel, eluting with petroleum ether/ethyl acetate (2:1 and 1:1).

Yield: 0.7 g (46% of theory),

R_{f} value: 0.38 (silica gel; petroleum ether/ethyl acetate=1:1)

b. -[1-(2-methyl-4-nitro-phenyl)-1,4,5,6-tetrahydro-cyclopentapyrazol-3-yl]-methyl methanesulphonate

0.8 g (2.9 mmol) of [1-(2-methyl-4-nitro-phenyl)-1,4,5,6-tetrahydro-cyclopentapyrazol-3-yl]-methanol are dissolved in 30 ml of tetrahydrofuran. After the addition of 0.6 ml (4.5 mmol) of triethylamine and 0.2 ml (3 mmol) of methanesulphonic acid chloride at 5° C. the reaction mixture is stirred for 2 hours at ambient temperature. After the addition of water the mixture is extracted with ethyl acetate, the combined organic extracts are dried and evaporated down.

Yield: 0.6 g (58% of theory),

R_{f} value: 0.65 (silica gel; petroleum ether/ethyl acetate=1:1)

### c. 4-(3-Dimethylaminomethyl-1,4,5,6-tetrahydro-cyclopentapyrazol-1-yl)-3-methyl-nitrobenzene

1.4 g (30.1 mmol) of dimethylamine are added dropwise to a solution of 0.9 g (2.5 mmol) of [1-(2-methyl-4-nitro-phenyl)-1,4,5,6-tetrahydro-cyclopentapyrazol-3-yl]-methyl methanesulphonate in 25 ml of tetrahydrofuran while cooling with ice. After 1 hour the solvent is distilled off, the residue is distributed in water/ethyl acetate and extracted. The combined organic extracts are dried over magnesium sulphate and evaporated down.

Yield: 0.6 g (72% of theory),

R_{f} value: 0.26 (silica gel; dichloromethane/ethanol=9:1+ammonia)

d. 4-(3-Dimethylaminomethyl-1,4,5,6-tetrahydro-cyclopentapyrazol-1-yl)-3-methyl-aniline

Prepared analogously to Example 1.c. from 4-(3-dimethylaminomethyl-1,4,5,6-tetrahydro-cyclopentapyrazol-1-yl)-3-methyl-nitrobenzene and palladium on activated charcoal/hydrogen in methanol.

Yield: 100% of theory,

R_{f} value: 0.3 (silica gel; dichloromethane/ethanol=9:1+ammonia)

e. 2-(2-benzyloxy-5-cyano-phenyl)-N-[4-(3-dimethylaminomethyl-1,4,5,6-tetrahydro -cyclopentapyrazol-1-yl)-3-methyl-phenyl]-acetamide

Prepared analogously to Example 1.h. from (2-benzyloxy-5-cyanophenyl)-acetic acid, 4-(3-dimethylaminomethyl-1,4,5,6-tetrahydro-cyclopentapyrazol-1-yl)-3-methyl-aniline, O-(benzotriazol-1-yl)-N,N,N′,N′-tetramethyluronium tetrafluoroborate and N-methylmorpholine in dimethylformamide.

Yield: 35% of theory,

R_{f} value: 0.45 (silica gel; dichloromethane/ethanol=9:1+1% ammonia)

f. 2-(5-amidino-2-hydroxy-phenyl)-N-[4-(3-dimethylaminomethyl-1,4,5,6-tetrahydro -cyclopentapyrazol-1-yl)-3-methyl-phenyl]-acetamide-hydrochloride

Prepared analogously to Example 3.f. from 2-(2-benzyloxy-5-cyano-phenyl)-N-[4-(3-dimethylaminomethyl-1,4,5,6-tetrahydro-cyclopentapyrazol-1-yl)-3-methyl-phenyl]-acetamide and hydrochloric acid/ammonium carbonate in ethanol followed by reaction with hydrogen/palladium on activated charcoal in methanol analogously to Example 1.c.

Yield: 92% of theory,

R_{f} value: 0.6 (Reversed phase RP 8; 5% sodium chloride solution/methanol=1:1) C₂₅H₃₀N₆O₂×HCl (446.56/483.02)

Mass spectrum: (M+H)⁺=447 (M+Cl)⁻=481/83 (Cl)

### EXAMPLE 8

### 2-(5-amidino-2-hydroxy-phenyl)-N-[4-(6,7-dihydro-5H-cyclopentapyrimidin-4-yl)-3-methyl-phenyl]-acetamide-hydrochloride

a. 4-(2-methyl-4-nitro-phenyl)-6,7-dihydro-5H-cyclopentapyrimidine

A mixture of 0.5 g (2.0 mmol) of 2-(2-methyl-4-nitro-benzoyl)-cyclopentanone and 2.1 g (20 mmol) of formamidine-acetate is heated to 150° C. for 2 hours. After cooling, the reaction mixture is stirred with water and extracted with ethyl acetate. The organic phase is washed with water and with saturated saline solution, dried and evaporated down. The residue is purified on silica gel, eluting with dichloromethane/methanol/conc. ammonia (100/0/0 to 98/1.95/0.05). The uniform fractions are combined and evaporated down.

Yield: 0.2 g (33% of theory)

R_{f} value: 0.31 (silica gel; petroleum ether/ethyl acetate=1:1)

b. 4-(2-methyl-4-amino-phenyl)-6,7-dihydro-5H-cyclopentapyrimidine

Prepared analogously to Example 1.c. from 4-(2-methyl-4-nitro-phenyl)-6,7-dihydro-5H-cyclopentapyrimidine and palladium on activated charcoal/hydrogen in methanol.

Yield: 100% of theory,

R_{f} value: 0.33 (silica gel; ethyl acetate+ammonia)

c. 2-(5-cyano-2-benzyloxy-2phenyl)-N-[4-(6,7-dihydro-5H-cyclopentapyrimidin-4-yl)-3-methyl-phenyl]-acetamide

Prepared analogously to Example 1.h. from (2-benzyloxy-5-cyanophenyl)-acetic acid, 4-(2-methyl-4-nitro-phenyl)-6,7-dihydro-5H-cyclopentapyrimidine, O-(benzotriazol-1-yl)-N,N,N′-N′-tetramethyluronium tetrafluoroborate and N-methylmorpholine in dimethylformamide.

Yield: 56% of theory,

R_{f} value: 0.5 (silica gel; dichloromethane/ethyl acetate=1:1+ammonia)

d. 2-(5-amidino-2-hydroxy-phenyl)-N-[4-(6,7-dihydro-5H-cyclopentapyrimidin-4-yl)-3-methyl-phenyl]-acetamide-hydrochloride

Prepared analogously to Example 3.f. from 2-(5-cyano-2-benzyloxy-phenyl)-N-[4-(6,7-dihydro-5H-cyclopentapyrimidin-4-yl)-3-methyl-phenyl]-acetamide and hydrochloric acid/ammonium carbonate in ethanol followed by reaction analogously to Example 1.c. with hydrogen/palladium on activated charcoal in methanol.

Yield: 59% of theory,

R_{f} value: 0.36 (Reversed phase RP 8; 5% sodium chloride solution/methanol=2:3) C₂₃H₂₃N₅O₂×HCl (401.25/437.93)

Mass spectrum: M+H)⁺=402 M−H)⁻=400

### EXAMPLE 9

### N-(5-amidino-2-hydroxy-benzyl)-3-trifluoromethyl-4-(3-methyl-1,4,5,6-tetrahydro-cyclopentapyrazol-1-yl)-benzamide-hydrochloride

a. (2-trifluoromethyl-4-cyano-phenyl)-hydrazine

Prepared analogously to Example 5a from 4-fluoro-3-trifluoromethyl-benzonitrile and hydrazine hydrate.

Yield: 89% of theory,

R_{f} value: 0.56 (silica gel; petroleum ether/ethyl acetate=4:1)

b. 3-methyl-1-(2-trifluoromethyl-4-cyano-phenyl)-1,4,5,6-tetrahydro-cyclopentapyrazole

Prepared analogously to Example 5b from 2-trifluoromethyl-4-cyano-phenylhydrazine and 2-acetyl-cyclopentanone.

Yield: 58% of theory,

R_{f} value: 0.15 (silica gel; dichloromethane)

c. 4-(3-methyl-1,4,5,6-tetrahydro-cyclopentapyrazol-1-yl)-3-trifluoromethyl-benzoic acid

0.8 g (2.7 mmol) of 1-methyl-3-(2-trifluoromethyl-4-cyano-phenyl)-1,4,5,6-tetrahydro-cyclopentapyrazole are suspended in 10 ml of 10N sodium hydroxide solution and after the addition of 10 ml of ethanol heated to 80° C. for 40 minutes. After the evaporation of the ethanol the residue is combined with water and filtered through activated charcoal. After the addition of 20 ml of 6N hydrochloric acid the precipitate formed is suction filtered, washed with water and dried.

Yield: 0.74 g (87% of theory),

R_{f} value: 0.13 (silica gel; cyclohexane/ethyl acetate=2:1+1% glacial acetic acid.

d. 4-benzyloxy-3-hydroxymethyl-benzonitrile

A solution of 1.7 g (6.9 mmol) of 4-benzyloxy-3-formyl-benzonitrile in 10 ml of tetrahydrofuran is added dropwise at 5-10° C. to a solution of 0.15 g (3.9 mmol) of sodium borohydride in 20 ml of tetrahydrofuran. After 1.5 hours at 10° C. the solvent is distilled off. The residue is combined with 0.5 N sodium hydroxide solution and extracted with ethyl acetate. The organic phase is dried, evaporated down and crystallised from with ether/petroleum ether.

Yield: 1.5 g (91% of theory),

R_{f} value: 0.2 (silica gel; petroleum ether/ethyl acetate=8:2)

e. 4-benzyloxy-3-(1,3-dioxo-1,3-dihydro-isoindol-2-yl)-methyl-benzonitrile

A solution of 2.6 g (15 mmol) of diethyl azodicarboxylate in 5 ml of tetrahydrofuran is added dropwise, at ambient temperature, to a solution of 0.9 g (6.2 mmol) of phthalimide-potassium salt, 1.5 g (6.2 mmol) of 4-benzyloxy-3-hydroxymethyl-benzonitrile and 3.9 g (15 mmol) of triphenylphosphine in 50 ml of tetrahydrofuran, during which time the temperature rises to 42° C. After 24 hours the solvent is distilled off, the residue is taken up in sodium chloride solution/ethyl acetate and extracted with ethyl acetate. The combined organic extracts are dried and chromatographed on silica gel, eluting with petroleum ether/ethyl acetate (10:0, 9:1 and 8:2).

Yield: 0.7 g (31% of theory),

R_{f} value: 0.45 (silica gel; petroleum ether/ethyl acetate=7:3)

f. 4-benzyloxy-3-aminomethyl-benzonitrile

0.7 g (1.9 mmol) of 4-benzyloxy-3-(1,3-dioxo-1,3-dihydro-isoindol-2-yl)-methyl-benzonitrile are dissolved in 20 ml of Isopropanol and refluxed for 30 minutes with the addition of 1.5 ml of hydrazine hydrate. Then the reaction solution is evaporated down, the residue is stirred with ice water, suction filtered and dried.

Yield: 0.3 g (71% of theory),

R_{f} value: 0.1 (silica gel; petroleum ether/ethyl acetate=1:1)

g. N-(5-cyano-2-benzyloxy-benzyl)-3-trifluoromethyl-4-(3-methyl-1,4,5,6-tetrahydro-cyclopentapyrazol-1-yl)-benzamide

Prepared analogously to Example 1.h. from 4-benzyloxy-3-aminomethyl-benzonitrile, 4-(3-methyl-1,4,5,6-tetrahydro-cyclopentapyrazol-1-yl)-3-trifluoromethyl-benzoic acid, O-(benzotriazol-1-yl)-N,N,N′,N′-tetramethyluronium tetrafluoroborate and N-methylmorpholine in dimethylformamide.

Yield: 59% of theory,

R_{f} value: 0.38 (silica gel; ethyl acetate+ammonia)

h. N-(5-amidino-2-hydroxy-benzyl)-3-trifluoromethyl-4-(3-methyl-1,4,5,6-tetrahydro-cyclopentapyrazol-1-yl)-benzamide-hydrochloride

Prepared analogously to Example 3.f. from N-(5-cyano-2-benzyloxy-benzyl)-3-trifluoromethyl-4-(3-methyl-1,4,5,6-tetrahydro-cyclopentapyrazol-1-yl)-benzamide and hydrochloric acid/ammonium carbonate in ethanol followed by reaction with hydrogen/palladium on activated charcoal in methanol analogously to Example 1.c.

Yield: 98% of theory,

R_{f} value: 0.23 (Reversed phase RP 8; 5% sodium chloride solution/methanol=2:3) C₂₃H₂₂F₃N₅O₂×HCl (457.47/493.92)

Mass spectrum: (M+H)⁺=458 (M−H)⁻=456 (M+Cl)⁻=492/4 (Cl)

### EXAMPLE 10

### N-(5-amidino-2-hydroxy-benzyl)3-trifluoromethyl-4-(3-methylaminocarbonyl-1,4,5,6-tetrahydro-cyclopentapyrazol-1-yl)-benzamide-hydrochloride

a. 4-hydrazino-3-trifluoromethyl-benzoic acid

Prepared analogously to Example 5.a. from 4-fluoro-3-trifluoromethyl-benzonitrile and hydrazine hydrate.

Yield: 79% of theory,

R_{f} value: 0.21 (silica gel; cyclohexane/ethyl acetate=2:1+1% glacial acetic acid)

b. 3-trifluoromethyl-4-(3-ethoxycarbonyl-1,4,5,6-tetrahydro-cyclopentapyrazol-1-yl)-benzoic acid

Prepared analogously to Example 5.b. from 4-hydrazino-3-trifluoromethyl-benzoic acid and ethyl 2-oxo-(2-oxo-cyclopentyl)-acetate.

Yield: 35% of theory

R_{f} value: 0.34 (silica gel; dichloromethane/ethanol=19:1+1% glacial acetic acid)

c. 3-trifluoromethyl-4-(3-methylaminocarbonyl-1,4,5,6-tetrahydro-cyclopentapyrazol-1 -yl)-benzoic acid

450 mg (1.2 mmol) of 3-trifluoromethyl-4-(3-ethoxycarbonyl-1,4,5,6-tetrahydro-cyclopentapyrazol-1-yl)-benzoic acid are combined with 4.5 ml of aqueous methylamine solution (40%) and heated to 140° C. for 1 hour. After cooling the reaction mixture is combined with ice water and glacial acetic acid. The solid formed is suction filtered, washed with ethyl acetate and ether and dried.

Yield: 290 mg (67% of theory)

R_{f} value: 0.30 (silica gel; ethyl acetate/ethanol=9:1+1% glacial acetic acid)

d. N-(5-cyano-2-benzyloxy-benzyl)-3-trifluoromethyl-4-(3-methylaminocarbonyl-1,4,5,6-tetrahydro-cyclopentapyrazol-1-yl)-benzamide

Prepared analogously to Example 9.g. from 4-benzyloxy-3-aminomethyl-benzonitrile, 3-trifluoromethyl-4-(3-methylaminocarbonyl-1,4,5,6-tetrahydro-cyclopentapyrazol-1 -yl)-benzoic acid, O-(benzotriazol-1-yl)-N,N,N′,N′-tetramethyluronium tetrafluoroborate and N-methylmorpholine in dimethylformamide.

Yield: 60% of theory,

R_{f} value: 0.66 (silica gel; ethyl acetate)

e. N-(5-amidino-2-hydroxy-benzyl)-3-trifluoromethyl-4-(3-methylaminocarbonyl-1,4,5,6-tetrahydro-cyclopentapyrazol-1-yl)-benzamide-hydrochloride

Prepared analogously to Example 3.f. from N-(5-cyano-2-benzyloxy-benzyl)-3-trifluoromethyl-4-(3-methylaminocarbonyl-1,4,5,6-tetrahydro-cyclopentapyrazol-1-yl)-benzamide and hydrochloric acid/ammonium carbonate in ethanol followed by reaction with hydrogen/palladium on activated charcoal in methanol analogously to Example 1.c.

Yield: 84% of theory,

R_{f} value: 0.66 (Reversed phase RP 8; 5% sodium chloride solution/methanol 1:3) C₂₄H₂₃F₃N₆O₃×HCl (500.49/536.95)

Mass spectrum: (M+H)⁺=501 (M+C)⁻=535/7 (Cl)

The following compounds are prepared analogously to Example 10:

### (1) N-(5-amidino-2-hydroxy-benzyl)-3-trifluoromethyl-4-(3-dimethylaminocarbonyl-1,4,5,6-tetrahydro-cyclopentapyrazol-1-yl)-benzamide-hydrochloride

Yield: 79% of theory,

R_{f} value: 0.65 (Reversed phase RP 8; 5% sodium chloride solution/methanol 1:3) C₂₅H₂₅F₃N₆O₃×HCl (514.51/550.98)

Mass spectrum: (M+H)⁺=515 (M−H)⁻=513

### (2) N-(5-amidino-2-hydroxy-benzyl)-3-trifluoromethyl-4-(3-methoxycarbonyl-1,4,5,6-tetrahydro-cyclopentapyrazol-1-yl)-benzamide-hydrochloride

Yield: 69% of theory,

R_{f} value: 0.65 (Reversed phase RP 8; 5% sodium chloride solution/methanol 1:3) C₂₄H₂₂F₃N₅O₄×HCl (501.47/537.94)

Mass spectrum: (M+H)⁺=502 (M−H)⁻=500 (M+Cl)⁻=536

### EXAMPLE 11

### ethyl 3-(3-amidino-phenyl)-3-[3-trifluoromethyl-4-(4,5,6,7-tetrahydro-benzimidazol-1-yl)-benzoylamino]-propionate-hydrochloride

a. 4-(4,5,6,7-tetrahydro-benzimidazol-1-yl)-3-methyl-benzonitrile

Prepared analogously to Example 4.a. from 3-trifluoromethyl-4-fluoro-benzonitrile and 4,5,6,7-tetrahydro-benzimidazole.

Yield: 88% of theory,

R_{f} value: 0.36 (silica gel; ethyl acetate)

b. 4-(4,5,6,7-tetrahydro-benzimidazol-1-yl)-3-methyl-benzoic acid

Prepared analogously to Example 9.c. from 4-(4,5,6,7-tetrahydro-benzimidazol-1-yl)-3-methyl-benzonitrile and sodium hydroxide solution.

Yield: 94% of theory,

R_{f} value: 0.38 (silica gel; ethyl acetate/ethanol=9:1+1% glacial acetic acid)

c. 3-amino-3-(3-cyano-phenyl)-propionic acid

13.1 g (0.1 mol) of 3-cyanobenzaldehyde are dissolved in 50 ml of 95% ethanol and after the addition of 15.4 g (0.2 mol) of ammonium acetate stirred for 15 minutes at 45° C. Then 20.8 g (0.2 mol) of malonic acid in 50 ml of 95% ethanol are added dropwise. The reaction mixture is refluxed for 2 hours. The crystalline product is suction filtered and recrystallised from methanol/water.

Yield: 6.5 g (34% of theory), C₁₀H₁₀N₂O₂ (190.20)

Mass spectrum: (M+H)⁺=191 (M−H)⁻=189

d. ethyl 3-amino-3-(3-cyano-1phenyl)-1propionate

0.9 g (4.8 mmol) of 3-amino-3-(3-cyano-phenyl)-propionic acid are suspended in 15 ml of ethanol and at 0° C. combined with a solution of 0.57 ml (4.8 mmol) of thionylchloride in 15 ethanol. The resulting solution is refluxed for 4 hours. The solvent is distilled off, the residue is taken up in 50 ml of ethyl acetate and extracted with 50 ml of saturated sodium hydrogen carbonate solution. The aqueous phase is extracted 3 times with 50 ml of ethyl acetate. The combined organic phases are washed with saturated saline solution, dried and evaporated down.

Yield: 0.78 g (75% of theory), light-coloured oil

R_{f} value: 0.5 (silica gel; dichloromethane/ethanol=19:1)

e. ethyl 3-(3-cyanophenyl)-3-[3-trifluoromethyl-4-(4,5,6,7-tetrahydro-benzimidazol-1-yl)-benzoylamino]-propionate

Prepared analogously to Example 1.h. from ethyl 3-amino-3-(3-cyano-phenyl)-propionate and 4-(4,5,6,7-tetrahydro-benzimidazol-1-yl)-3-methyl-benzoic acid.

Yield: 88% of theory,

R_{f} value: 0.36 (silica gel; ethyl acetate)

f. ethyl 3-(3-amidino-phenyl)-3-[3-trifluoromethyl-4-(4,5,6,7-tetrahydro-benzimidazol-1-yl)-benzoylamino]-propionate-hydrochloride

Prepared analogously to Example 3.f. from ethyl 3-(3-cyanophenyl)-3-[3-trifluoromethyl-4-(4,5,6,7-tetrahydro-benzimidazol-1-yl)-benzoylamino]-propionate and hydrochloric acid/ammonium carbonate in ethanol.

Yield: 60% of theory,

R_{f} value: 0.3 (silica gel; dichloromethane/ethanol 4:1+1% glacial acetic acid) C₂₇H₂₈F₃N₅O₃×HCl (527.55/564.02)

Mass spectrum: (M−H)⁻=526 (M+Cl)⁻=562/4 (Cl)

### EXAMPLE 12

### 3-(3-amidino-phenyl)-3-[3-trifluoromethyl-4-(4,5,6,7-tetrahydro-benzimidazol-1-yl)-benzoylamino]-propionic acid-hydrochloride

320 mg (0.57 mmol) of ethyl 3-(3-amidino-phenyl)-3-[3-trifluoromethyl-4-(4,5,6,7-tetrahydro-benzimidazol-1-yl)-benzoylamino]-propionate hydrochloride are dissolved in 10 ml of 6N hydrochloric acid and stirred for 12 hours at ambient temperature. The solvent is evaporated down with the addition of toluene, the residue is stirred in 60 ml of ethyl acetate/acetone/diethylether (1:1:1). The precipitate formed is filtered off and dried.

Yield: 220 mg (72% of theory),

R_{f} value: 0.4 (silica gel; dichloromethane/ethanol=4:1+1% glacial acetic acid) C₂₅H₂₄F₃N₅O₃×HCl (499.5/535.97)

Mass spectrum: (M+H)⁺=500 (M−H)⁻=498 (M+Cl)⁻=534/6 (Cl)

The following compounds are prepared analogously to Example 12:

### (1) 2-(3-amidino-phenyl)-3-13-trifluoromethyl-4-(3-aminomethyl-1,4,5,6-tetrahydro-cyclopentapyrazol-1-yl)-benzoylamino]-propionic acid]-dihydrochloride

Yield: 87% of theory,

R_{f} value: 0.61 (Reversed phase RP 8; 5% sodium chloride solution/methanol=1:2) C₂₅H₂₅F₃N₆O₃×2HCl (514.51/587.43)

Mass spectrum: (M+H)⁺=515 (M−H)⁻=513

### (2) 2-(3-aminomethyl-phenyl)-3-[3-trifluoromethyl-4-(3-aminomethyl-1,4,5,6-tetrahydro-cyclopentapyrazol-1-yl)-benzoylamino]-propionic acid]-dihydrochloride

Yield: 92% of theory,

R_{f} value: 0.51 (Reversed phase RP 8; 5% sodium chloride solution/methanol=1:1) C₂₅H₂₆F₃N₅O₃×2HCl (501.51/574.43)

Mass spectrum: (M−H)⁻=500 (M+Cl)⁻=536/8

### EXAMPLE 13

### N-(5-amidino-2-hydroxy-benzyl)-3-trifluoromethyl-4-(3-tert.butyloxycarbonylaminomethyl-1,4,5,6-tetrahydro-cyclopentapyrazol-1-yl)-benzamide

a. 3-Dimethoxymethyl-1,4,5,6-tetrahydro-cyclopentapyrazole

Prepared analogously to Example 6.b. from 2-(2,2-dimethoxy-acetyl)-cyclopentanone and hydrazine.

Yield: 56% of theory C₉H₁₄N₂O₂ (182.22)

Mass spectrum: (M+H)⁺=183 (M+Na)⁺=205

b. 3-Dimethoxymethyl-1-(2-trifluoromethyl-4-cyano-phenyl)-1,4,5,6-tetrahydro-cyclopentapyrazole

0.7 g (17.5 mmol) of sodium hydride are added at 0° C. to a solution of 3.2 g (17 mmol) of 4-fluoro-3-trifluoromethyl-benzonitrile and 3.1 g (17 mmol) of 3-dimethoxymethyl-1,4,5,6-tetrahydro-cyclopentapyrazole in 20 ml of dimethylformamide. After 3 hours at ambient temperature the reaction solution is poured onto ice water and extracted with ethyl acetate. The organic phase is washed with water and with saturated saline solution, dried and evaporated down. The crude product is recrystallised from diisopropylether/petroleum ether.

Yield: 3.2 g (54% of theory)

R_{f} value: 0.33 (silica gel; petroleum ether/ethyl acetate=7:3+1% conc. ammonia)

c. 3-Formyl-1-(2-trifluoromethyl-4-cyano-phenyl)-1,4,5,6-tetrahydro-cyclopentapyrazole

4.7 ml of sulphuric acid (15%, 12.2 mmol) are added to a suspension of 32 g of silica gel in 100 ml of dichloromethane. Then a solution of 4.3 g (12.2 mmol) of 3-dimethoxymethyl-1-(2-trifluoromethyl-4-cyano-phenyl)-1,4,5,6-tetrahydro-cyclopentapyrazole in 75 ml of dichloromethane is added. After 20 minutes at ambient temperature the silica gel is filtered off and the filtrate is evaporated down. The crude product is further reacted without any purification.

Yield: 3.7 g (99% of theory)

R_{f} value: 0.27 (silica gel; petroleum ether/ethyl acetate=7:3+1% conc. ammonia)

d. 3-tert.butyloxycarbonylaminomethyl-1-(2-trifluoromethyl-4-cyano-phenyl)-1,4,5,6-tetrahydro-cyclopentapyrazole

4.3 g (36.4 mmol) of tert.butylcarbamate, 5.8 ml (36.4 mmol) of triethylsilane and 1.9 ml (24.2 mmol) of trifluoracetic acid are added to a solution of 3.7 g (12.1 mmol) of 3-formyl-1-(2-trifluoromethyl-4-cyano-phenyl)-1,4,5,6-tetrahydro-cyclopentapyrazole in 60 ml of acetonitrile at ambient temperature. After 20 hours at ambient temperature the reaction mixture is taken up in ether, washed with saturated sodium hydrogen carbonate solution and with saturated saline solution, dried and evaporated down.

Yield: 4.9 g (100% of theory)

R_{f} value: 0.2 (silica gel; petroleum ether/ethyl acetate=7:3+1% conc. ammonia)

e. 4-(3-tert.butyloxycarbonylaminomethyl -1,4,5,6-tetrahydro-cyclopentapyrazol-1-yl)-3-trifluoromethyl-benzoic acid

Prepared analogously to Example 9.c. from 3-tert.butyloxycarbonylaminomethyl-1-(2-trifluoromethyl-4-cyano-phenyl)-1,4,5,6-tetrahydro-cyclopentapyrazole and 10N sodium hydroxide solution.

Yield: 89% of theory

R_{f} value: 0.22 (silica gel; dichloromethane/methanol=19:1+1% glacial acetic acid)

f. N-(5-cyano-2-benzyloxy-benzyl)-3-trifluoromethyl-4-(3 -tert.butyloxycarbonylaminomethyl-1,4,5,6-tetrahydro-cyclopentapyrazol-1-yl)-benzamide

Prepared analogously to Example 1.h. from 4-benzyloxy-3-aminomethyl-benzonitrile, 4-(3-tert.butyloxycarbonylaminomethyl-1,4,5,6-tetrahydro-cyclopentapyrazol-1-yl)-3-trifluoromethyl-benzoic acid, O-(benzotriazol-1-yl)-N,N,N′,N′-tetramethyluronium tetrafluoroborate and N-methylmorpholine in dimethylformamide.

Yield: 86% of theory,

R_{f} value: 0.4 (silica gel; ethyl acetate/petroleum ether 1:1)

g. N-(5-N′-hydroxy-amidino-2-benzyloxy-benzyl)-3-trifluoromethyl-4-(3-tert.butyloxycarbonylaminomethyl-1,4,5,6-tetrahydro-cyclopentapyrazol-1-yl)-benzamide

Prepared analogously to Example 1.h. from N-(5-cyano-2-benzyloxy-benzyl)-3-trifluoromethyl-4-(3-tert.butyloxycarbonylaminomethyl-1,4,5,6-tetrahydro-cyclopentapyrazol-1-yl)-benzamide and hydroxylamine.

Yield: 66% of theory,

R_{f} value: 0.23 (silica gel; ethyl acetate/petroleum ether =1:2+1% ammonia)

h. N-(5-amidino-2-hydroxy-benzyl)-3-trifluoromethyl-4-(3-tert.butyloxycarbonylaminomethyl-1,4,5,6-tetrahydro-cyclopentapyrazol-1-yl)-benzamide

Prepared analogously to Example 1.c. from N-(5-N′-hydroxy-amidino-2-benzyloxy-benzyl)-3-trifluoromethyl-4-(3-tert.butyloxycarbonylaminomethyl-1,4,5,6-tetrahydro-cyclopentapyrazol-1-yl)-benzamide and hydrogen/palladium (20% on activated charcoal).

Yield: 75% of theory,

R_{f} value: 0.36 (silica gel; dichloromethane/methanol=4:1+1% ammonia) C₂₈H₃₁F₃N₆O₄ (572.59)

Mass spectrum: (M+H)⁺=573 (M−H)⁻=571

### EXAMPLE 14

Dry ampoule containing 75 mg of active substance per 10 ml

| Composition: | | |
|---|---|---|
| Active substance | 75.0 | mg |
| Mannitol | 50.0 | mg |
| water for injections ad | 10.0 | ml |

Preparation

Active substance and mannitol are dissolved in water. After packaging the solution is freeze-dried. To produce the solution ready for use, the product is dissolved in water for injections.

### EXAMPLE 15

Dry ampoule containing 35 mg of active substance per 2 ml

| Composition: | | |
|---|---|---|
| Active substance | 35.0 | mg |
| Mannitol | 100.0 | mg |
| water for injections ad | 2.0 | ml |

Preparation

Active substance and mannitol are dissolved in water. After packaging, the solution is freeze-dried.

To produce the solution ready for use, the product is dissolved in water for injections.

### EXAMPLE 16

Tablet containing 50 mg of active substance

| | |
|---|---|
| Composition: | |
| (1) Active substance | 50.0 mg |
| (2) Lactose | 98.0 mg |
| (3) Maize starch | 50.0 mg |
| (4) Polyvinylpyrrolidone | 15.0 mg |
| (5) Magnesium stearate | 2.0 mg |
| | 215.0 mg |

Preparation

(1), (2) and (3) are mixed together and granulated with an aqueous solution of (4). (5) is added to the dried granulated material. From this mixture tablets are pressed, biplanar, faceted on both sides and with a dividing notch on one side. Diameter of the tablets: 9 mm.

### EXAMPLE 17

Tablet containing 350 mg of active substance

| | |
|---|---|
| Composition: | |
| (1) Active substance | 350.0 mg |
| (2) Lactose | 136.0 mg |
| (3) Maize starch | 80.0 mg |
| (4) Polyvinylpyrrolidone | 30.0 mg |
| (5) Magnesium stearate | 4.0 mg |
| | 600.0 mg |

Preparation

(1), (2) and (3) are mixed together and granulated with an aqueous solution of (4). (5) is added to the dried granulated material. From this mixture tablets are pressed, biplanar, faceted on both sides and with a dividing notch on one side.

Diameter of the tablets: 12 mm.

### EXAMPLE 18

Capsules containing 50 mg of active substance

| | |
|---|---|
| Composition: | |
| (1) Active substance | 50.0 mg |
| (2) Dried maize starch | 58.0 mg |
| (3) Powdered lactose | 50.0 mg |
| (4) Magnesium stearate | 2.0 mg |
| | 160.0 mg |

Preparation

(1) is triturated with (3). This trituration is added to the mixture of (2) and (4) with vigorous mixing.

This powder mixture is packed into size 3 hard gelatine capsules in a capsule filling machine.

### EXAMPLE 19

Capsules containing 350 mg of active substance

| | |
|---|---|
| Composition: | |
| (1) Active substance | 350.0 mg |
| (2) Dried maize starch | 46.0 mg |
| (3) Powdered lactose | 30.0 mg |
| (4) Magnesium stearate | 4.0 mg |
| | 430.0 mg |

Preparation

(1) is triturated with (3). This trituration is added to the mixture of (2) and (4) with vigorous mixing.

This powder mixture is packed into size 0 hard gelatine capsules in a capsule filling machine.

### EXAMPLE 20

Suppositories containing 100 mg of active substance

| | |
|---|---|
| 1 suppository contains: | |
| Active substance | 100.0 mg |
| Polyethyleneglycol (M.W. 1500) | 600.0 mg |
| Polyethyleneglycol (M.W. 6000) | 460.0 mg |
| Polyethylenesorbitan monostearate | 840.0 mg |
| | 2,000.0 mg |

Preparation

The polyethyleneglycol is melted together with polyethylenesorbitan monostearate. At 40° C. the ground active substance is homogeneously dispersed in the melt. This is then cooled to 38° C. and poured into slightly chilled suppository moulds.

What is claimed is:

1. A compound of the formula I 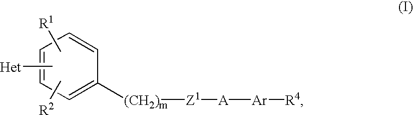
wherein:
m denotes the number 0 or 1,
A denotes a straight-chain C₁₋₃-alkylene group wherein
one or two hydrogen atoms independently of one another may each be replaced by a C₁₋₃ alkyl group or
a hydrogen atom may be replaced by the group —(CH₂)ₚ—R_{f}, wherein
p denotes one of the numbers 0,1,2 or 3 and
R_{f} denotes a hydroxycarbonyl, C₁₋₃-alkoxycarbonyl, aminocarbonyl, C₁₋₃-alkylaminocarbonyl, C₁₋₃-(dialkyl)-aminocarbonyl or
C₃₋₇-cycloalkylamino-carbonyl group,
Ar denotes a phenylene or naphthylene group optionally substituted by a fluorine, chlorine or bromine atom, by a carboxy, carboxy-C₁₋₃-alkyl, carboxy-C₁₋₃-alkoxy, alkoxycarbonyl-C₁₋₃-alkoxy, trifluoromethyl, C₁₋₃-alkyl, hydroxy, C₁₋₃-alkoxy, phenyl-C₁₋₃-alkoxy, amino, C₁₋₃-alkylamino or di-(C₁₋₃-alkyl)-amino group, wherein the phenylene group may be substituted by another fluorine, chlorine or bromine atom or by another C₁₋₃-alkyl group; or
a thienylene, thiazolylene, pyridinylene, pyrimidinylene, pyrazinylene or pyridazinylene group optionally substituted in the carbon skeleton by a C₁₋₃-alkyl group,
Het denotes an imidazolyl, pyrazolyl, pyridyl or pyrimidyl group optionally substituted by a substituent selected from among the C₁₋₃-alkyl, hydroxy, C₁₋₃-alkylaminocarbonyl or C₁₋₃-alkoxycarbonyl group and the group of formula
RₐR_{b}N—(CH₂)ₒ,
wherein
Rₐ and R_{b} independently of one another each denote a hydrogen atom or a C₁₋₃-alkyl group, or Rₐ and R_{b} taken together form a C₂₋₅-alkylenediyl group, and
o denotes 0 or 1,
wherein a phenyl or C₅₋₆-cycloalkyl ring is fused to this group via two adjacent carbon atoms and the bicyclic heterocyclyl groups thus formed are bound via the heterocyclic moiety,
Z¹ denotes a —CO—NR³ or —NR³—CO— group;
R¹ denotes a hydrogen, fluorine, chlorine or bromine atom, a hydroxy group or a C₁₋₃-alkyl or C₁₋₃-alkoxy group optionally substituted by one or more fluorine atoms,
R² denotes a hydrogen atom or a C₁₋₃-alkyl group,
R³ denotes a hydrogen atom or a C₁₋₃-alkyl group optionally substituted by a carboxy group, and
R⁴ denotes a cyano group, an aminomethyl group or an amidino group optionally substituted by one or two C₁₋₃-alkyl groups
wherein the carboxy groups mentioned in the definition of the abovementioned groups may be replaced by a group which may be converted in vivo into a carboxy group or by a group which is negatively charged under physiological conditions, or
the amino, imino and amidino groups mentioned in the definition of the abovementioned groups may be substituted by a group which can be cleaved in vivo,
or a tautomer or salt thereof.

2. A compound of the formula I according to claim 1, wherein:
m denotes the number 0 or 1,
A denotes a methylene group wherein
one or two hydrogen atoms independently of one another may each be replaced by a C₁₋₃ alkyl group or a hydrogen atom may be replaced by the group —(CH₂)ₚ—R_{f}, wherein
p denotes one of the numbers 0,1,2 or 3 and
R_{f} denotes a hydroxycarbonyl, C₁₋₃-alkoxycarbonyl, aminocarbonyl, C₁₋₃-alkylaminocarbonyl, C₁₋₃-(dialkyl)-aminocarbonyl or C₃₋₇-cycloalkylamino-carbonyl group,
Ar denotes a phenylene group optionally substituted by a fluorine, chlorine or bromine atom, by a methyl, hydroxy, methoxy or benzyloxy group which may be substituted by another methyl group;
Het denotes an imidazolyl, pyrazolyl, pyridyl or pyrimidyl group optionally substituted by a substituent selected from among the C₁₋₃-alkyl, hydroxy, C₁₋₃-alkylaminocarbonyl or C₁₋₃-alkoxycarbonyl group and the group of formula
RₐR_{b}N—(CH₂)ₒ,
wherein
Rₐ and R_{b} independently of one another each denote a hydrogen atom or a C₁₋₃-alkyl group, or Rₐ and R_{b} taken together form a C₂₋₅-alkylenediyl group, and
o denotes 0 or 1,
wherein a phenyl or C₅₋₆-cycloalkyl ring is fused to this group via two adjacent carbon atoms and the bicyclic heterocyclyl groups thus formed are bound via the heterocyclic moiety,
Z¹ denotes a —CO—NR³ or —NR³—CO— group;
R¹ denotes a hydrogen, fluorine, chlorine or bromine atom, a methyl, trifluoromethyl, difluoromethyl, hydroxy or methoxy group,
R² denotes a hydrogen atom or a methyl group,
R³ denotes a hydrogen atom or a methyl or ethyl group optionally substituted by a carboxy or C₁₋₃-alkoxycarbonyl group, and
R⁴ denotes a cyano group, an aminomethyl group or an amidino group,
or a tautomer or salt thereof.

3. A compound of the formula I, according to claim 1, wherein:
m denotes the number 0,
A denotes a methylene group wherein
a hydrogen atom may be replaced by a C₁₋₃ alkyl group or by the group —(CH₂)ₚ—R_{f}, wherein
p denotes one of the numbers 0,1,2 or 3 and
R_{f} denotes a hydroxycarbonyl, C₁₋₃-alkoxycarbonyl, aminocarbonyl, C₁₋₃-alkylaminocarbonyl or C₁₋₃-(dialkyl)-aminocarbonyl group,
Ar denotes a phenylene group optionally substituted by a methyl, hydroxy, methoxy or benzyloxy group,
Het denotes an imidazolyl, pyrazolyl, pyridyl or pyrimidyl group optionally substituted by a substituent selected from among the C₁₋₃-alkyl, hydroxy, C₁₋₃-alkylaminocarbonyl or C₁₋₃-alkoxycarbonyl group and the group of formula
RₐR_{b}N—(CH₂)ₒ,
wherein
Rₐ and R_{b} independently of one another each denote a hydrogen atom or a C₁₋₃-alkyl group, or Rₐ and R_{b} taken together form a C₂₋₅-alkylenediyl group, and
o denotes 0 or 1,
wherein a phenyl or C₅₋₆-cycloalkyl ring is fused to this group via two adjacent carbon atoms and the bicyclic heterocyclyl groups thus formed are bound via the heterocyclic moiety,
Z¹ denotes a —CO—NR³ or —NR³—CO— group;
R¹ denotes a hydrogen, fluorine, chlorine or bromine atom or a methyl or trifluoromethyl group,
R² denotes a hydrogen atom,
R³ denotes a hydrogen atom or a methyl or ethyl group substituted by a carboxy, methoxycarbonyl or ethoxycarbonyl group, and
R⁴ denotes an aminomethyl or amidino group,
or a tautomer or salt thereof thereof.

4. A compound of the formula I, according to claim 3, wherein —Ar—R⁴ denotes a 5-amidino-2-hydroxyphenyl group.

5. A compound of the formula IA, 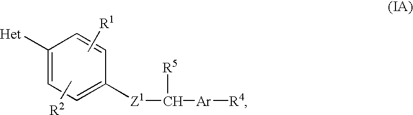
wherein: Ar denotes a phenylene or naphthylene group optionally substituted by a fluorine, chlorine or bromine atom, by a carboxy, carboxy-C₁₋₃-alkyl, carboxy-C₁₋₃-alkoxy, alkoxycarbonyl-C₁₋₃-alkoxy, trifluoromethyl, C₁₋₃-alkyl, hydroxy, C₁₋₃-alkoxy, phenyl-C₁₋₃-alkoxy, amino, C₁₋₃-alkylamino or di-(C₁₋₃-alkyl)-amino group, wherein the phenylene group may be substituted by another fluorine, chlorine or bromine atom or by another C₁₋₃-alkyl group; or
a thienylene, thiazolylene, pyridinylene, pyrimidinylene, pyrazinylene or pyridazinylene group optionally substituted in the carbon skeleton by a C₁₋₃-alkyl group,
Het denotes a 5 or 6-membered heterocyclic group optionally substituted by one, two or three substituents and bound via a carbon or nitrogen atom, wherein these substituents are selected from among ═O, a fluorine, chlorine or bromine atom, a trifluoromethyl, C₁₋₃-alkyl, hydroxy, hydroxy-C₁₋₃-alkyl, C₁₋₃-alkoxy, phenyl-C₁₋₃-alkoxy, C₁₋₃-alkylaminocarbonyl, C₁₋₃-dialkylaminocarbonyl, C₃₋₇-cycloalkylaminocarbonyl, C₁₋₃-alkylaminocarbonyl-(CH₂)ₒ—C₁₋₃-alkoxycarbonyl or C₁₋₃-alkoxycarbonyl-(CH₂)ₒ— group and the group of formula
RₐR_{b}N—(CH₂)ₒ,
wherein
Rₐ and R_{b} independently of one another each denote a hydrogen atom or a C₁₋₃-alkyl, aminocarbonyl, C₁₋₄-alkyloxycarbonyl, amidino, C₁₋₃-alkylCOHNC(NH), C₁₋₃-alkylC(NH), imidazol-2-yl or 4,5-dihydroimidazol-2-yl— group, or Rₐ and R_{b} taken together form a C₂₋₅-alkylenediyl group, and
o denotes 0 or 1,
wherein a phenyl or C₄₋₈-cycloalkyl ring may be fused to the abovementioned 5- or 6-membered heterocyclic group via two adjacent cyclic atoms and the bicyclic heterocyclyl groups thus formed may be bound via the heterocyclic or carbocyclic moiety,
Z¹ denotes a —CO—NR³ or —NR³—CO— group;
R¹ denotes a hydrogen, fluorine, chlorine or bromine atom, a hydroxy group or a C₁₋₃-alkyl or C₁₋₃-alkoxy group optionally substituted by one or more fluorine atoms,
R² denotes a hydrogen atom or a C₁₋₃-alkyl group,
R³ denotes a hydrogen atom or a C₁₋₃-alkyl group optionally substituted by a carboxy group,
R⁴ denotes a cyano group, an aminomethyl group or an amidino group optionally substituted by one or two C₁₋₃-alkyl groups, and
R⁵ denotes a hydrogen atom, a C₁₋₃ alkyl group or a group of formula —(CH₂)ₚ—R_{f}, wherein
p denotes one of the numbers 0,1,2 or 3 and
R_{f} denotes a hydroxycarbonyl, C₁₋₃-alkoxycarbonyl, aminocarbonyl, C₁₋₃-alkylaminocarbonyl, C₁₋₃-(dialkyl)-aminocarbonyl or C₃₋₇-cycloalkylamino-carbonyl group.

6. A compound selected from the group consisting of:
2-(5-amidino-2-hydroxy-phenyl)-N-[3-methyl-4-(2-tert-butoxycarbonylaminomethyl-benzimidazol-1-yl)-phenyl]-acetamide;
2-(5-amidino-2-hydroxy-phenyl)-N-[3-methyl-4-(2-aminomethyl-benzimidazol-1-yl)-phenyl]-acetamide;
2-(5-amidino-2-hydroxy-phenyl)-N-[4-(4,5-dimethyl-2-oxo-2,3-dihydroimidazol-1-yl)-3-methyl-phenyl]-acetamide;
2-(5-amidino-2-hydroxy-phenyl)-N-[4-(benzimidazol-1-yl)-3-methyl-phenyl]-acetamide;
2-(5-amidino-2-hydroxy-phenyl)-N-[3-methyl-4-(2-methyl-benzimidazol-1-yl)-phenyl]-acetamide;
2-(5-amidino-2-hydroxy-phenyl)-N-[3-methyl-4-((2-pyrrolidin-1-yl)-benzimidazol-1-yl)-phenyl]-acetamide;
2-(5-amidino-2-hydroxy-phenyl)-N-[3-methyl-4-(2-dimethylamino-benzimidazol-1-yl)-phenyl]-acetamide;
2-(5-amidino-2-hydroxy-phenyl)-N-[3-methyl-4-(4,5,6,7-tetrahydro-benzimidazol-1-yl)-phenyl]-acetamide;
2-(5-amidino-2-hydroxy-phenyl)-N-[3-methyl-4-(3-ethoxycarbonyl-1,4,5,6-tetrahydro-cyclopentapyrazol-1-yl)-phenyl]-acetamide;
2-(5-amidino-2-hydroxy-phenyl)-N-[4-(3-methyl-1,4,5,6-tetrahydro-cyclopentapyrazol-1-yl)-phenyl]-acetamide;
N-(5-amidino-2-hydroxy-benzyl)-3-methyl-4-(3-methyl-1,4,5,6-tetrahydro-cyclopentapyrazol-1-yl)]-benzamide;
2-(5-amidino-2-hydroxy-phenyl)-N-[3-methyl-4-(1-methyl-1,4,5,6-tetrahydro-cyclopentapyrazol-3-yl)-phenyl]-acetamide;
2-(5-amidino-2-hydroxy-phenyl)-N-[3-methyl-4-(2-methyl-2,4,5,6-tetrahydro-cyclopentapyrazol-3-yl)-phenyl]-acetamide;
2-(5-amidino-2-hydroxy-phenyl)-N-[3-methyl-4-(1,4,5,6-tetrahydro-cyclopentapyrazol-3-yl)-phenyl]-acetamide;
2-(5-amidino-2-hydroxy-phenyl)-N-[4-(3-dimethylaminomethyl-1,4,5,6-tetrahydro-cyclopentapyrazol-1-yl)-3-methyl-phenyl]-acetamide;
2-(5-amidino-2-hydroxy-phenyl)-N-[4-(6,7-dihydro-5H-cyclopentapyrimidin-4-yl)-3-methyl-phenyl]-acetamide;
N-(5-amidino-2-hydroxy-benzyl)-3-trifluoromethyl-4-(1-methyl-1,4,5,6-tetrahydro-cyclopentapyrazol-3-yl)-benzamide;
N-(5-amidino-2-hydroxy-benzyl)-3-trifluoromethyl-4-(3-methylaminocarbonyl-1,4,5,6-tetrahydro-cyclopentapyrazol-1-yl)-benzamide;
2-(5-amidino-2-hydroxy-phenyl)-N-[3-trifluoromethyl-4-(4,5,6,7-tetrahydro-benzimidazol-1-yl)-phenyl]-acetamide;
2-(5-amidino-2-hydroxy-phenyl)-N-[3-trifluoromethyl-4-(4,5,6,7-tetrahydro-benzimidazol-1-yl)-phenyl]-propionamide;
N-(5-amidino-2-hydroxy-benzyl)-N-3-trifluoromethyl-4-(4,5,6,7-tetrahydro-benzimidazol-1-yl)-benzamide; and
N-[1-(5-amidino-2-hydroxy-phenyl)-ethyl]-3-trifluoromethyl-4-(4,5,6,7-tetrahydro-benzimidazol-1-yl)-benzamide,
or a salt thereof.

7. A physiologically acceptable salt of a compound according to claim 1, 2, 3, 4 or 5, wherein R⁴ denotes an amidino group, or a physiologically acceptable salt of a compound according to claim 6.

8. A pharmaceutical composition comprising a compound according to claim 1, 2, 3, 4 or 5, wherein R⁴ denotes an amidino group, or a compound according to claim 6, or a physiologically acceptable salt thereof, an a pharmaceutically acceptable carrier or diluent.

10. A method for treating thrombosis which comprises administering to a host in need of such treatment an antithrombotic amount of a compound according to claim 1, 2, 3, 4 or 5, wherein R⁴ denotes an amidino group, or a compound according to claim 6, or a physiologically acceptable salt thereof.
